(19) United States
(12) Patent Application Publication
(10) Pub. No.: US 2013/0011875 A1
(43) Pub. Date: Jan. 10, 2013

(54) METHODS FOR THE PRODUCTION OF RECOMBINANT PROTEINS WITH IMPROVED SECRETION EFFICIENCIES

(75) Inventors: Michael Meehl, Lebanon, NH (US); Heping Lin, West Lebanon, NH (US); Byung-Kwon Choi, Norwich, VT (US)

(73) Assignee: Merck Sharpe & Dohme Corp, Rahway (NJ)

(21) Appl. No.: 13/503,707

(22) PCT Filed: Oct. 25, 2010
(86) PCT No.: PCT/US10/53903

§ 371 Date: Oct. 1, 2012

Related U.S. Application Data
(60) Provisional application No. 61/256,379, filed on Oct. 30, 2009.
(60) Provisional application No. 61/350,668, filed on Jun. 2, 2010.

(51) Int. Cl. C12N 1/19; C12P 21/00

(52) U.S. Cl. 435/69.1; 435/254.23

(57) ABSTRACT

The present invention is related to methods and for producing higher titers of recombinant protein in a modified yeast host cell, for example Pichia pastoris, wherein the modified yeast cell lacks vacuolar sorting activity or has decreased vacuolar sorting activity relative to an unmodified yeast host cell of the same species. In particular embodiments vacuolar sorting activity is reduced or eliminated by deletion or disruption of a gene encoding Vps10 or a Vps10 homolog. The invention is also related to the modified yeast cells which are modified in accordance with the methods disclosed herein.

### CROSS REFERENCE TO RELATED APPLICATIONS

This application claims the benefit of U.S. Provisional Application No. 61/256,379, filed Oct. 30, 2009, and U.S. Provisional Application No. 61/350,668, filed Jun. 2, 2010, the disclosures of which are herein incorporated by reference in their entirety.

### FIELD OF THE INVENTION

The invention relates to methods and compositions for producing recombinant proteins in fungal cells, including yeast cells, with increased secretion efficiencies.

### REFERENCE TO SEQUENCE LISTING SUBMITTED ELECTRONICALLY

The sequence listing of the present application is submitted electronically via EFS-Web as an ASCII formatted sequence listing with a file name “GFIMIS00004_SEQTXT_{—}18OCT2010.TXT”, creation date of Oct. 18, 2010, and a size of 861 KB. This sequence listing submitted via EFS-Web is part of the specification and is herein incorporated by reference in its entirety.

### BACKGROUND OF THE INVENTION

Expression of recombinant proteins in eukaryotic cells has become increasingly important due to the current focus on biologic therapeutics, which represents the largest growth segment of FDA-regulated drugs. Whether the production cell is a CHO-based mammalian cell line or glycoengineered Pichia pastoris (Sethuraman and Stadheim, Curr. Opin. Biotechnol. 17: 341-346 (2006)), maximal secretion titers are critical. While many efforts to increase protein production focus on promoter and copy number of the recombinant gene (Daly and Hearn, J. Mol. Recognit. 18: 1999-38 (2005)), efficient secretion is only achieved if the recombinant protein transits a specific path from the endoplasmic reticulum (ER) to the Golgi apparatus, followed by the trans-Golgi network and finally, to the exocytic vesicles for delivery through the plasma membrane. If the recombinant protein deviates from this desired secretory route, the yield will decline.

Glycoengineered yeast offer distinct advantages for therapeutics development compared to mammalian cells. For example, the glycosylation profiles of mammalian cell-based systems are heterogeneous (Li et al., Nat. Biotechnol. 24: 210-15 (2006)) while glycoengineered Pichia pastoris has proven to provide uniform glycosylation (Hamilton et al., Science 313:1441-43 (2006)). Although genetic modifications of mammalian glycosylation are possible, such as eliminating fucose (Shinkawa et al., J. Biol. Chem. 278: 3466-73 (2003)), most glycoform selection must occur at the fermentation and/or purification steps, often limiting yield. The ease of genetic manipulations in yeast affords opportunities to improve protein yield independent of fermentation and purification compared to mammalian host cells.

In yeast, endogenous proteins that are delivered to the vacuole are degraded by proteinases. The yeast vacuole is an organelle analogous to the mammalian lysosome that is critically important for endocytosis, protein turnover, and nutrient acquisition to maintain cellular homeostasis. One mechanism of vacuolar protein trafficking is the carboxypeptidase Y pathway, which delivers proteins from the trans Golgi network (TGN). In Saccharomyces cerevisiae, the protein receptors responsible for initial interactions of carboxypeptidase Y in the TGN are named Vps10 (also known as Pep1 or Vpt1), Vth1, and Vth2. In S. cerevisiae, Vps10 functions to deliver vacuolar-residing proteinases to the prevacuolar compartment, leading to eventual proteolysis in the vacuole (for reviews, see Bowers and Stevens, Biochim. Biophys. Acta 1744:438-54 (2005); Li and Kane, Biochim. Biophys. Acta. 1983: 650-663 (2009), epub August 2008).

Marcusson et al. (Cell 77: 579-586 (1994)) showed that in Saccharomyces cerevisiae, Vps10 is required for the sorting of Cpy to the yeast vacuole. Marcusson et al. further showed that mutation of the VPS10 gene leads to defective vacuolar protein sorting of endogenous Cpy, leading to its secretion. However, it was also shown that disruption of VPS10 and loss of Vps 10 activity did not have any affect on the sorting of the vacuolar enzymes PrA and PrB, which properly transited the path to the vacuole in a S. cerevisiae strain in which the VPS10 gene was knocked-out. Iwaki et al. (Microbiology 152: 1523-32 (2006)) also showed that deletion of VPS10 in Schizosaccharomyces pombe resulted in missorting and secretion of Cpy, suggesting that Vps10 is required for sorting Cpy to the vacuole. The Vps10 sorting receptor was also shown to function in Cpy sorting in a similar fashion for Saccharomyces pombe (Takegawa et al., Curr Genet. 42(5):252-9 (2003); Iwaki et al., Microbiology 152(5):1523-32 (2006)).

J. Denecke (U.S. Patent Application No. 2005/0019855) discloses a method of limiting proteolysis by preventing export of proteins out of the ER and/or redirecting proteins from the vacuolar sorting pathway back to the ER or the cell surface. It is further suggested that the vacuolar sorting receptor Vps10 can be modified in such a way to re-direct proteins back to the ER, thereby increasing heterologous protein expression.

Idiris et al. (Appl Microbial. Biotechnol. 85(3):667-77 (2010), Epub 2009 Aug. 11) describe a 2-fold increased secretion of human growth hormone (hGH) in the strain A8-vps10Δ, which is a Schizosaccharomyces pombe strain that comprises a VPS10 deletion as well as eight protease gene deletions, when compared to the A8 strain that had only the eight protease deletions. However, a low level of r-hGH secretion was retained intracellularly, which suggested that several VPS genes, which are related to intracellular protein retention, must be deleted in order to completely block the vacuolar accumulation pathway.

Takegawa et al. (supra) also describe a vps10 deficient strain of Schizosacharomyces pombe and show that Cpy is not processed to its mature form in this mutant. However, this study does not describe the expression of heterologous therapeutic protein in the vps10Δ strain.

Agaphonov et al. (FEMS Yeast Research 5: 1029-1035 (2005)) inactivated the VPS10 gene in Hansenula polymorpha and did not observe an increase in secretion of human urokinase-type plasminogen activator (uPA). In this study, an increase in proteolytic processing of uPA was observed in the VPS10 deficient strain.

It would be highly desirable to develop methods of increasing the yield of heterologous proteins produced in fungal or yeast cells by eliminating or reducing vacuolar sorting activity.

### SUMMARY OF THE INVENTION

The present invention is related to, inter alia, methods for producing a recombinant protein in a yeast or fungal host cell comprising: (a) transforming a genetically modified yeast or fungal host cell with an expression vector encoding the protein to produce a host cell, wherein the genetically modified yeast or fungal cell lacks vacuolar sorting activity or has decreased vacuolar sorting activity relative to an unmodified yeast or fungal host cell of the same species; (b) culturing the transformed yeast or fungal host cell in a medium under conditions which induce expression of the protein in fermentation conditions; and (c) isolating the protein from the transformed yeast or fungal host cell or culture medium. In some embodiments of this aspect of the invention, the yeast or fungal host cell is selected from the group consisting of: Pichia pastoris, Saccharomyces cerevisiae, Aspergillus niger, Saccharomyces pombe, Candida albicans, Candida glabrata, Pichia stipitis, Debaryomyces hansenii, Kluyveromyces lactic, and Hansenula polymorpha (also known as Pichia angusta). In one preferred embodiment, the host cell is a Pichia cell, in specific embodiments the host cell is Pichia pastoris.

In other embodiments, the invention relates to a method for producing a recombinant protein in a yeast or fungal host cell comprising: (a) expressing the recombinant protein in a genetically modified yeast or fungal host cell, wherein the genetically modified yeast or fungal host cell lacks vacuolar sorting activity or has decreased vacuolar sorting activity relative to an unmodified yeast or fungal host cell of the same species; (b) culturing the genetically modified yeast or fungal host cell in a medium under conditions which induce expression of the protein in fermentation conditions; and (c) isolating the protein from the yeast or fungal host cell or culture medium.

In particular embodiments of the methods of the invention, vacuolar sorting activity is eliminated or reduced by deletion or disruption of a gene encoding Vps10 or a Vps10 homolog such as Vps10-1 from the fungal or yeast cell genome.

The invention also relates to a method for producing a recombinant protein in a Pichia host cell comprising: (a) transforming a genetically modified Pichia cell with an expression vector encoding the protein to produce a host cell, wherein the genetically modified Pichia cell lacks vacuolar sorting activity relative to an unmodified Pichia cell of the same species; (b) culturing the transformed Pichia host cell in a medium under conditions that induce expression of the protein; and (c) isolating the protein from the transformed cell or culture medium. In some embodiments of this aspect of the invention, the host cell is a Pichia pastoris cell.

The invention further provides a Pichia pastoris cell lacking vacuolar sorting activity or having reduced vacuolar sorting activity relative to a wild-type Pichia pastoris cell, wherein the host cell comprises a functional deletion of a vacuolar protein sorting receptor 10-1 (Vps10-1), for example the Vps10-1 protein set forth in SEQ ID NO:20. In some embodiments, the P. pastoris cell is further modified to express glycoproteins in which the glycosylation pattern is human-like. In still further embodiments, a gene encoding Vps10-1 is deleted and a gene encoding Vps10-2 is intact (i.e., not deleted).

As used throughout the specification and in the appended claims, the singular forms “a,” “an,” and “the” include the plural reference unless the context clearly dictates otherwise.

As used throughout the specification and appended claims, the following definitions and abbreviations apply:

### DEFINITIONS

“QRPL-like’ sorting signal” refers to a vacuolar sorting signal that allows a recombinant protein to bind to Vps10. In carboxypeptidase Y (Cpy), the sequence QRPL (SEQ ID NO:176) binds to Vps10, leading to Cpy being directed to the vacuole. “QRPL-like” sorting signals have homology to the QRPL sequence and allow binding of the recombinant protein to Vps10 or a Vps10 homolog. Examples of “QRPL-like” sorting signals include, but are not limited to, “QSFL” (SEQ ID NO:179) and “QVAF” (SEQ ID NO:180).

“Vps10-1” refers to a vacuolar sorting receptor 10-1 in a Pichia pastoris cell, such as the Vps10-1 protein as defined by the amino acid sequence set forth in SEQ ID NO:20. One skilled in the art will realize that minor variations in Vps10-1 sequence can occur in different Pichia pastoris cell lines that will not alter the function of the protein. Thus, a reference to Vps10-1 includes the protein sequence set forth in SEQ ID NO:2 and protein sequences that are structurally and functionally similar, i.e. function in an equivalent manner (e.g. participate in vacuolar sorting) and have an amino acid sequence with at least 90% sequence identity to SEQ ID NO:20, more preferably at least 92% identity, at least 94% identity, even more preferably at least 96% identity, at least 98% identity or at least 99% identity.

“Vps10-2” refers to a vacuolar sorting receptor 10-2 in a Pichia pastoris cell, such as the Vps10-2 protein as defined by the amino acid sequence set forth in SEQ ID NO:21 One skilled in the art will realize that minor variations in Vps 10-2 sequence can occur in different Pichia pastoris cell lines that will not alter the function of the protein. Thus, a reference to Vps10-2 includes the protein sequence set forth in SEQ ID NO:21 and protein sequences that are structurally and functionally similar, i.e. function in an equivalent manner and have an amino acid sequence with at least 90% identity to SEQ ID NO:21, more preferably at least 92% identity, at least 94% identity, even more preferably at least 96% identity or at least 98% identity.

“Homolog,” as used herein, refers to a gene or protein sequence that shares structural and functional similarity to a reference sequence. The term “homolog” includes both orthologs, which are sequences in different species that are structurally similar due to evolution from a common ancestor, and paralogs, which are similar sequences within the same genome.

“Reduction of protein function” including “reduced vacuolar sorting activity” refers to the reduction of protein function in a “modified” host cell relative to a host cell of the same species that does not comprise the modification at issue. The function of a particular protein is said to be “reduced” when the modified protein has at least 20% to 50% lower activity, in particular aspects, at least 40% lower activity or at least 50% lower activity, when measured in a standard assay, relative to an unmodified protein. One skilled in the art understands that both the “modified host cell” and the “unmodified host cell” may comprise additional mutations that are not related to the protein which is being functionally assessed. For example, when assessing reduction of Vps 10 protein function, a “modified” Pichia pastoris host cell which comprises a deletion of Vps10 and further comprises a deletion of BMT1 so as to eliminate glycoproteins having α-mannosidase-resistant N-glycans is compared to an “unmodified” host cell which does not comprise a Vps 10 deletion, but does comprise a BMT1 deletion.

“Elimination of protein function” refers to the elimination of protein function or activity in a “modified” host cell relative to a host cell of the same species which does not comprise the modification to the particular protein being assessed. In particular embodiments, a modified protein is said to have “eliminated function” when it has at least 90% to 99% lower activity relative to a protein without said modification. In particular aspects, the modified protein has at least 95% lower activity or at least 99% lower activity, when measured in a standard assay. In some aspects the modified protein has completely ablated protein activity or function.

The term “deleted or disrupted” and “deletion or disruption” or “functional deletion” as used herein refers to any disruption or inhibition of the activity or function of a particular protein, such as the Pichia pastoris Vps10-1 and Vps10-2 proteins, Vps10 homologs in other species such as Saccharomyces cerevisiae, or other proteins which participate in vacuolar sorting, said protein produced from a yeast cell genome, in which the inhibition of the protein activity renders the protein incapable of performing its intended function or only capable of performing its intended function to a lesser degree relative to an unmodified yeast cell of the same species not comprising the deletion or disruption. Examples of which are yeast host cells in which vacuolar sorting activity can be abrogated or disrupted including, but not limited to, 1) deletion or disruption of the upstream or downstream regulatory sequences controlling expression of a gene which participates in vacuolar sorting; 2) mutation of the gene encoding the protein activity to render the gene non-functional, where “mutation” includes deletion, substitution, insertion, or addition into the gene to render the encoded protein incapable of vacuolar sorting activity; 3) abrogation or disruption of the vacuolar sorting activity by means of a chemical, peptide, or protein inhibitor; 4) abrogation or disruption of the vacuolar sorting activity by means of nucleic acid-based expression inhibitors, such as antisense RNA, RNA interference, and siRNA; 5) abrogation or disruption of the vacuolar sorting activity by means of transcription inhibitors or inhibitors of the expression or activity of regulatory factors that control or regulate expression of the gene encoding the enzyme activity; 6) co-expression of a peptide or protein that is known to bind to Vps 10, such as Cpy, to saturate the vacuolar receptor and reduce sorting of secreted recombinant protein; 7) co-expression of a mutated Vps10 protein that is not membrane associated or a dominant-negative Vps 10 protein that acts to prevent normal vacuolar sorting patterns; 8) alteration of the amino acid sequence of the recombinant protein of interest to eliminate a Vps10-binding domain and prevent vacuolar sorting; and 9) by any means in which the protein product obtained, even if expressed, is not identical to the protein obtained from an unmodified yeast cell and the function is attenuated.

### ABBREVIATIONS

VPS10-1 vacuolar protein sorting receptor 1
VPS10-2 vacuolar protein sorting receptor 2
ScSUC2 S. cerevisiae invertase
OCH1 alpha-1,6-mannosyltransferase
KlMNN2-2: K. lactis UDP-GlcNAc transporter
BMT1: beta-mannose-transfer 1 (beta-mannose elimination)
BMT2: beta-mannose-transfer 2 (beta-mannose elimination)
BMT3: beta-mannose-transfer 3 (beta-mannose elimination)
BMT4: beta-mannose-transfer 4 (beta-mannose elimination)
MNN4L1: MNN4-like 1 (charge elimination)
MmSLC35A3 mouse homologue of UDP-GlcNAc transporter
PNO1: phosphomannosylation of N-linked oligosaccharides (charge elimination)
MNN4: mannosyltransferase (charge elimination)
FB53: MmMNS1A fused to ScMNN2 leader
TrMDS1: secreted T. reseei MNS1
Sh ble: zeocin resistance marker
HSAss: human serum albumin signal sequence
DAP2: dipeptidyl aminopeptidase
STE13: dipeptidyl aminopeptidase
CLP1: P. pastoris cellulase-like protein 1
5-FOA 5-fluoroorotic acid
TNFRII-Fc tumor necrosis factor receptor 2 ectodomain fused to Fc region of IgG1
ER endoplasmic reticulum
GCSF granulocyte colony-stimulating factor
rhGCSF recombinant human granulocyte colony-stimulating factor

### BRIEF DESCRIPTION OF THE DRAWINGS

FIG. 1 shows the construction of pGLY5192 (vps10-1 knock-out plasmid) and pGLY5194 (vps10-2 knock-out plasmid). Plasmid maps of constructs that were used to generate pGLY5192 and pGLY5194, including restriction enzyme sites and insert DNA, are shown.

FIGS. 2A-2B show the construction of plasmid vector pGLY5178 (rhGCSF expression plasmid) encoding rHuMetGCSF and targeting the Pichia pastoris AOX1 locus. Plasmid maps of constructs that were used to generate pGLY5178, including restriction enzyme sites and insert DNA, are shown.

FIG. 3 shows the construction of pGLY3465 (TNFRII-Fc expression plasmid). Plasmid maps, restriction enzymes, and insert DNA that were used to generate pGLY3465 are described.

FIGS. 4A-4E depict the generation of yGLY8538, a glycoengineered Pichia pastoris strain expressing rhGCSF. Strain construction involved the use of a parental strain and genetic alteration (via plasmid or media selection) to generate a resulting strain with the correct genotype, as listed. The annotation of genes listed in the genotype is described in the summary of the invention. The final strain, yGLY8538, is a recombinant human granulocyte colony-stimulating factor (rhGCSF) expression strain that was used to make subsequent mutant strains.

FIGS. 5A-5D depict the generation of yGLY9993. Strain construction involved the use of a parental strain and genetic alteration (via plasmid or media selection) to generate a resulting strain with the correct genotype, as listed. The annotation of genes listed in the genotype is described in the summary of the invention. The final strains, yGLY9992 and yGLY9993, are isogenic vps10-1 mutants of yGLY8292. These strains are zeocin sensitive and therefore do not contain rhGCSF or TNFRII-Fc.

FIG. 6 depicts the generation of yGLY8538 mutant strains. The rhGCSF expression strain yGLY8538 was mutated in genes vps10-1 (yGLY9933), vps10-2 (yGLY10566), or both (yGLY10557). Strain construction involved the use of a parental plasmid and genetic alteration (via plasmid or media selection) to generate a resulting strain with the correct genotype, as listed in relation to yGLY8538.

FIG. 7 shows the effect of Vps 10 activity on rhGCSF titer (Panel A) and cell lysis (Panel B). See Example 14. Data listed were generated from Sixfors (0.5L) fermentation experiments. Panel A: The listed strains were fermented under identical conditions and cell-free supernatant fluids were analyzed by ELISA to quantitate levels of rhGCSF. The ELISA values for each were divided by the parental control yGLY8538 ELISA value to obtain the relative titer. Panel B: The listed strains were fermented under identical conditions and cell-free supernatant fluids were analyzed by PicoGreen® assay to quantitate levels of double-stranded DNA. The PicoGreen® dsDNA values for each were divided by the parental control yGLY8538 PicoGreen® dsDNA value to obtain a relative cell lysis value.

FIG. 8 shows the effect of Vps 10 activity on TNFRII-Fc titer (see EXAMPLE 15). Data listed was generated from a 96 well deep well induction plate experiment. The listed strains were transformed with pGLY3465 and data represents relative titers from at least eleven independent colonies. Cell-free supernatant fluids were analyzed by ELISA to quantitate levels of TNFRII-Fc. The ELISA values for each parental strain were averaged then divided by the average ELISA value of parental control yGLY8292 to obtain the relative titer. Both yGLY9992 and yGLY9993 strains are independent mutants of vps10-1.

FIGS. 9A-B show a model of Vps10-activity in Pichia pastoris. Schematic diagrams of Vps 10 receptor functions in both wild-type (panel A) and vps10-1Δ mutant (panel B) strains. After mRNA transcription in the nucleus, the protein polypeptide is translated and translocated to the lumen of the endoplasmic reticulum. After transiting to the late Golgi, GCSF interacts with Vps10-1 in wild-type cells (A). Vps10-1, via a cytoplasmic tail, circulated from the Golgi to the prevacuolar compartment (PVC), where GCSF dissociates from the receptor. Whereas Vps10-1 circulates back to the Golgi, GCSF in the PVC migrates to the vacuole and is proteolytically degraded. In the mutant cell (B), Vps 10-1 protein is absent and therefore more GCSF is secreted to the culture supernatant fraction.

FIG. 10 lists the primer sequences used to generate plasmids described in the Examples (SEQ ID NOs: 1-13).

FIG. 11 lists the plasmids (panel A) and the strains (panel B) used in the Examples.

FIG. 12 provides a comparison of the length, percent similarity and percent identity between fungal Vps10 homologs, when compared to S. cerevisiae Vps10.

FIGS. 13A-13E show the nucleotide sequence of the Pichia pastoris VPS10-1 region (SEQ ID NO:14) including upstream homologous fragment, promoter, open reading frame (nucleotides 1610-6238), and downstream homologous fragment.

FIGS. 14A-14D show the nucleotide sequence of the Pichia pastoris VPS10-2 region (SEQ ID NO:15) including upstream homologous fragment, promoter, open reading frame (nucleotides 830-4509), and downstream homologous fragment.

FIG. 15 shows the amino acid sequence of P. pastoris Vps10-1 (SEQ ID NO:20).

FIG. 16 shows the amino acid sequence of P. pastoris Vps 10-2 (SEQ ID NO:21).

FIG. 17 shows the amino acid sequence of S. cerevisiae Vps 10 (also known as Pep1 or Vpt1, SEQ ID NO:22).

FIG. 18 shows the amino acid sequence of Aspergillus niger Vps10 (SEQ ID NO:26).

FIG. 19 shows the amino acid sequence of Saccharomyces pombe Vps10 (SEQ ID NO:27).

FIG. 20 shows the amino acid sequence of Candida albicans Vps10 (SEQ ID NO:28).

FIG. 21 shows the amino acid sequence of Candida glabrata Vps 10 (SEQ ID NO:29).

FIG. 22 shows the amino acid sequence of Pichia stipitis Vps 10 (SEQ ID NO:30).

FIG. 23 shows the amino acid sequence of Debaryomyces hansenii Vps10 (SEQ ID NO:181).

FIG. 24 shows the amino acid sequence of Kluyveromyces lactis Vps10 (SEQ ID NO:182).

FIG. 25 provides the SEQ ID NOs of the amino acid sequences of proteins associated with the CPY vacuolar sorting pathway.

FIG. 26 provides the SEQ ID NOs of the amino acid sequences of proteins associated with the recycling of Vps10 to the late Golgi from the PVC.

FIG. 27 provides the SEQ ID NOs of the amino acid sequences of proteins associated with proper MVB function and/or fusion to the vacuole.

FIG. 28 provides the SEQ ID NOs of the amino acid sequences of proteins that are associated with proper Cpy vacuolar targeting through unknown mechanisms.

### DETAILED DESCRIPTION OF THE INVENTION

The present invention provides, inter alfa, methods for producing recombinant proteins in a genetically modified yeast or fungal host cell lacking vacuolar sorting activity or having decreased vacuolar sorting activity relative to an unmodified yeast or fungal host cell of the same species, wherein the yeast or fungal cell is modified so as to eliminate the function of Saccharomyces cerevisiae Vps10, or a Vps10 homolog, including, but not limited to, Pichia pastoris Vps10-1. In some embodiments of the invention, the yeast or fungal cell is modified so that the gene encoding Vps10 or Vps10 homolog is deleted or disrupted, as described infra.

Efficient, high-yield expression of recombinant proteins in eukaryotic cells is essential to the development of many biologic therapeutic products. In order to achieve the high yield of proteins that is required for the commercial development of a therapeutic protein, it is important that maximal secretion titers of the protein are obtained. The secretory path of S. cerevisiae is well characterized with a large number of gene functions elucidated. After mRNA molecules are translated and proteins enter the ER lumen, numerous processes may occur to the protein including additions of asparagine-linked glycans (N-linked), serine/threonine-linked mannose (O-linked), folding assisted by ER-resident chaperones, disulfide bond formation, retro-translocation out of the ER, binding to cargo receptors, trafficking to the Golgi via COPII vesicles, and others.

It is a goal of the present invention to increase the titer of heterologously expressed therapeutic proteins in yeast cell culture, including yeast cell culture in fermentation conditions. The secretion of heterologously expressed proteins via exocytosis is negatively impacted by alternative trafficking to the vacuole. Vacuolar sorting of recombinant proteins could decrease the secretory yield in the supernatant fraction. In order to develop methods for increasing the secretion of recombinant proteins expressed in yeast or fungal cells, we initially considered modification of three potential alternative trafficking pathways, which may direct recombinant proteins to the vacuole: (1) cytoplasm-to-vacuole targeting (CVT), (2) the alkaline phosphatase pathway (ALP) (Piper et al. J Cell Biol 138: 531-45 (1997)), and (3) the carboxypeptidase Y (CPY) pathway (Marcusson et al., supra, and Cooper & Stevens, J Cell Biol 133: 529-41 (1996)). CVT is a specific type of autophagy whereby the normal cellular function is to direct vacuolar-resident proteins from the cytoplasm, after protein synthesis, to the vacuole. However, this pathway does not typically interact with recombinant proteins destined for the secretory pathway; therefore, it did not represent an opportunity to increase protein yield. The ALP pathway delivers membrane-bound proteins, such as alkaline phosphatase, in the Golgi to the vacuole via specific signaling interactions in the carboxy-terminal cytoplasmic domain of the membrane-bound ALP substrate. Since this pathway only sorts transmembrane proteins to the vacuole, which are typically not recombinant therapeutic proteins, it also did not represent a mechanism to increase secretory yield for therapeutic protein production.

The third alternative sorting mechanism in Saccharomyces cerevisiae, the CPY pathway, is a process by which pro-carboxypeptidase y (pro-Cpy, also known as Prc1) interacts with the vacuolar protein sorting receptor, Vps10 (also known as Pep1 or Vpt1), in the late Golgi. By way of vesicle trafficking mediated by numerous proteins with the carboxy-terminal cytoplasmic domain of Vps 10, pro-Cpy is targeted to an intermediate compartment named the prevacuolar complex (PVC) (also known as multivesicular body (MVB)). After dissociation of pro-Cpy from Vps 10 in the PVC, Vps 10 is recycled back to the late Golgi by a specific group of proteins. PVC vesicles containing pro-Cpy then are trafficked to the vacuole and a fusion event occurs with additional protein components. Pro-Cpy then matures to active Cpy in the vacuole and the sorting is completed. Of the three pathways initially considered, the CPY pathway is the most relevant to soluble, secreted recombinant proteins. Since recombinant proteins in the secretory pathway transit the late Golgi prior to exocytosis, they have the potential to interact with Vps10. Should a recombinant protein contain a sequence that binds to Vps10, the recombinant protein would be sorted to the vacuole or lysosome via the CPY pathway and likely degraded by proteases, thus reducing the secretion rate and limiting titer. We hypothesized that by eliminating vacuolar sorting through this pathway, more recombinant protein could be secreted via exocytosis, thereby increasing cell productivity.

Although much was known about the secretory pathway in S. cerevisiae for endogenous proteins, it was not known prior to the present invention whether the titer of a heterologously-expressed recombinant therapeutic protein could be improved by expressing a gene encoding the heterologous protein in a vps10 yeast mutant in fermentation conditions. It was also not known if a functional deletion of a vps10 homolog in a Pichia cell could increase the secretion of a recombinant protein encoded by a gene contained within an expression vector in the cell.

To this end, embodiments of the present invention are related to the identification of a major bottleneck of recombinant protein expression in yeast. As described above, in Saccharomyces cerevisiae, Vps10 is responsible for binding pro-Cpy and localizing the protein to the vacuole. Two homologs of the VPS10 gene were identified in Pichia pastoris, named VPS10-1 and VPS10-2. Vectors to create null mutations in the two loci, vps10-1 and vps10-2, were constructed. Plasmids were transformed in P. pastoris to create null mutants of these genes. The vps10-1 genetic mutants displayed increased secretion of rh-GCSF and TNFRII-Fc, The vps10-2 knock-out strain did not lead to increased secretion of rhGCSF and, for this reason, TNFRII-Fc secretion was not tested in this strain. Our data indicates both rhGCSF and TNFRII-Fc are targeted to the vacuole for degradation via Vps10-1 binding in the trans-Golgi network (TGN) of Pichia pastoris. Thus, it is demonstrated herein that in a Pichia host cell, a portion of a recombinantly-expressed protein is re-routed from the correct secretory pathway to an alternate pathway that leads to the yeast vacuole, via Vps10 interactions (Marcusson et al., Cell 77: 579-86 (1994)). Once proteins are sorted to the vacuole or lysosome, they are removed from the secretory pathway and are degraded by proteases, thus reducing the secretion rate of recombinant proteins. It is shown herein that by eliminating vacuolar sorting through the CPY pathway, more recombinant protein is secreted via exocytosis, thereby increasing cell productivity. In accordance with embodiments of the invention, it has been shown that genetic inactivation of a Pichia pastoris VPS10 homolog, VPS10-1, dramatically increased secretion of recombinant hGCSF and TNFRII-Fc into the culture medium. From the known amino acid sequences of GCSF and TNFRII-Fc, sequences were identified near the amino termini of these proteins with high homology to the “QRPL” consensus Vps10 binding sequence (see EXAMPLE 13, van Voorst et al., J. Biol. Chem. 271: 841-6 (1996)). Further, the reported crystal structure of these proteins (Hill et al., Proc. Natl. Acad. Sci. USA 90: 5167-71 (1993), Tamada et al. Proc. Acad. Sci. USA 103: 3135-40 (2006)) indicated that they contain surface-exposed peptides. These observations led to the development of methods described herein, in which secretory rates of recombinant proteins comprising “QRPL”-like sequences,” which bind to the vacuolar protein sorting receptor Vps10, can be improved via genetic alterations of VPS10 or a VPS10 homolog in the host cell of choice.

Thus, embodiments of the present invention provide methods for producing a recombinant protein in a yeast host cell comprising: (a) transforming a genetically modified fungal or yeast host cell with an expression vector encoding the protein to produce a host cell, wherein the genetically modified fungal or yeast cell lacks vacuolar sorting activity or has decreased vacuolar sorting activity relative to an unmodified fungal or yeast host cell of the same species; (b) culturing the transformed host cell in a medium under conditions which induce expression of the protein in fermentation conditions; and (c) isolating the protein from the transformed host cell or culture medium.

The invention also provides a method for producing a recombinant protein in a yeast or fungal host cell, the method comprising: (a) expressing the recombinant protein in a genetically modified yeast or fungal host cell, wherein the genetically modified yeast or fungal host cell lacks vacuolar sorting activity or has decreased vacuolar sorting activity relative to an unmodified yeast or fungal host cell of the same species; (b) culturing the genetically modified yeast or fungal host cell in a medium under conditions which induce expression of the protein in fermentation conditions; and (c) isolating the protein from the yeast or fungal host cell or culture medium.

In embodiments of the methods of the invention described above, the host cell is a yeast cell. In specific embodiments, the host cell is a Pichia cell, such as Pichia pastoris.

The invention further provides methods for producing a recombinant protein in a Pichia host cell comprising: (a) transforming a genetically modified Pichia cell with an expression vector encoding the protein to produce a host cell, wherein the genetically modified Pichia cell lacks vacuolar sorting activity or has decreased vacuolar sorting activity relative to an unmodified Pichia cell of the same species; (b) culturing the transformed Pichia host cell in a medium under conditions that induce expression of the protein; and (c) isolating the protein from the transformed host cell or culture medium.

In particular embodiments of this aspect of the invention, the host cell is a Pichia pastoris cell.

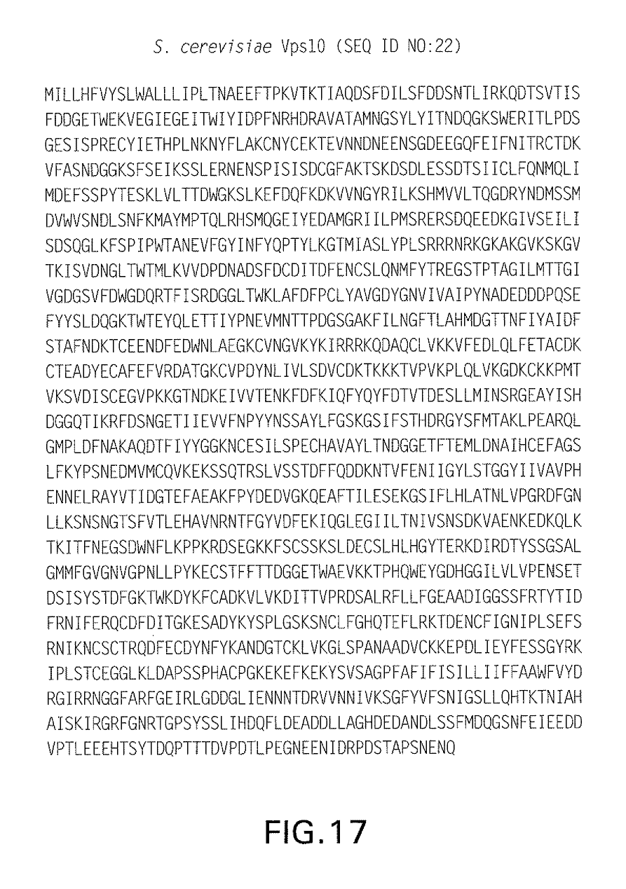

In accordance with the methods of the invention described above, vacuolar sorting activity can be eliminated or reduced from the host cell of choice by genetic deletion or disruption of a gene encoding Vps10 or a Vps10 protein homolog. In this embodiment of the invention, a Vps 10 protein homolog is identified in the desired host cell by, for example, using a known Vps10 or a known Vps10 protein homolog sequence to search the appropriate yeast or fungal genome using a computational search program such as TBLASTN, which searches for similar proteins in a translated nucleotide database (see Example 3). One skilled in the art may also identify VPS10 gene homologs in the desired host cell by designing PCR primers or DNA probes based on the known sequence of S. cerevisiae VPS10 and screening a DNA library comprising DNA of the desired host. The S. cerevisiae Vps10 amino acid sequence is shown in FIG. 17 (SEQ ID NO:22). Once a Vps10 protein homolog is identified in the desired host cell, vacuolar sorting activity can be functionally deleted from that host cell through deletion or disruption of the VPS10 gene homolog, as described herein.

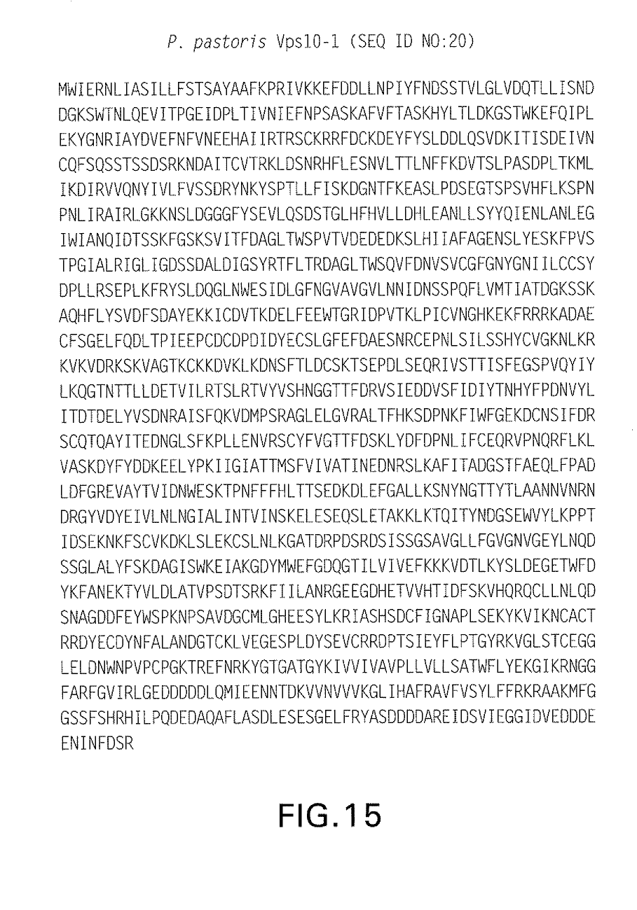

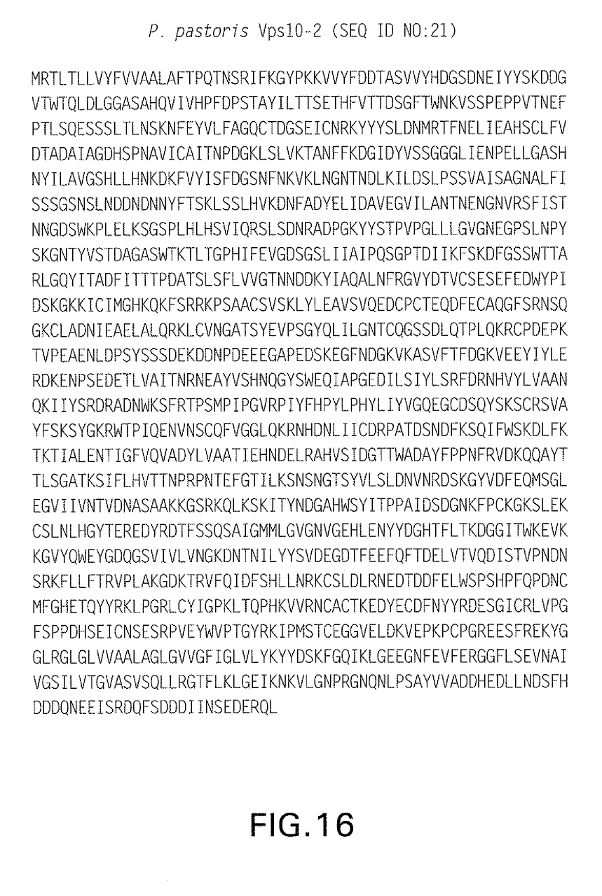

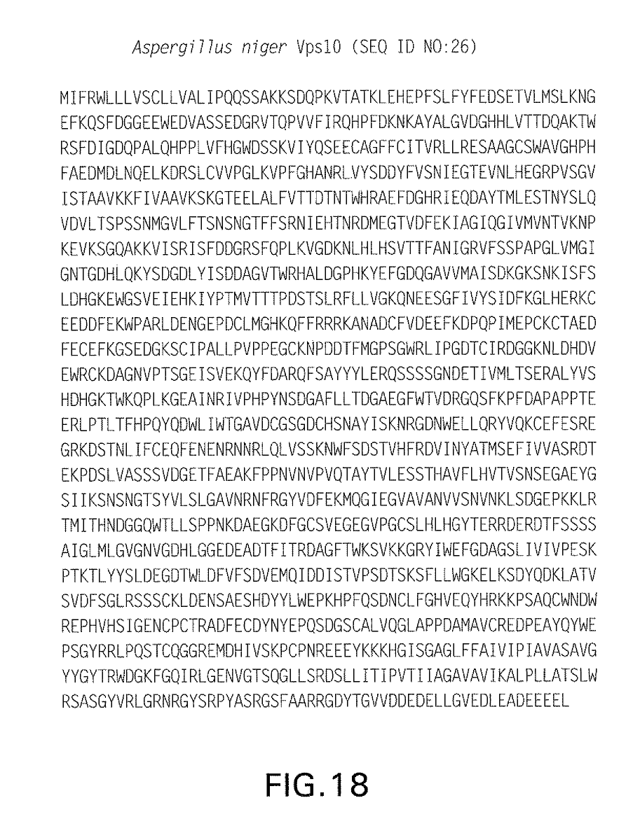

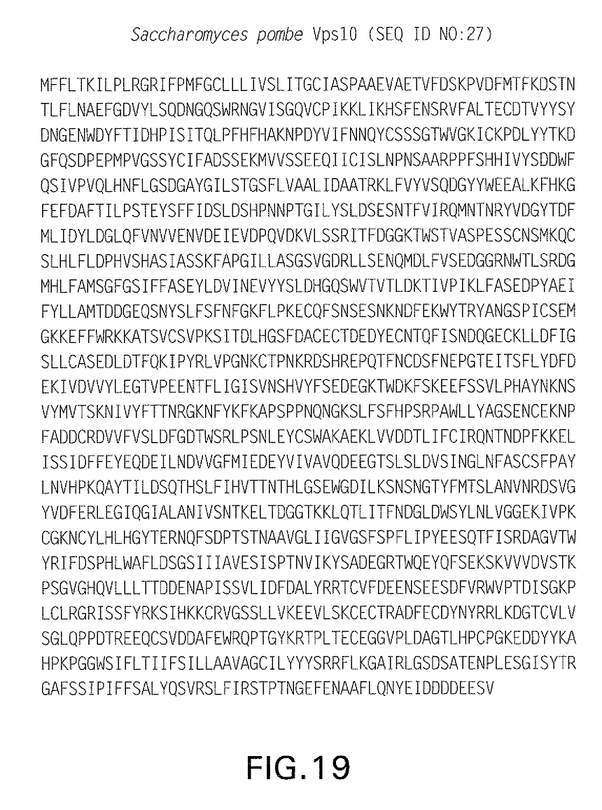

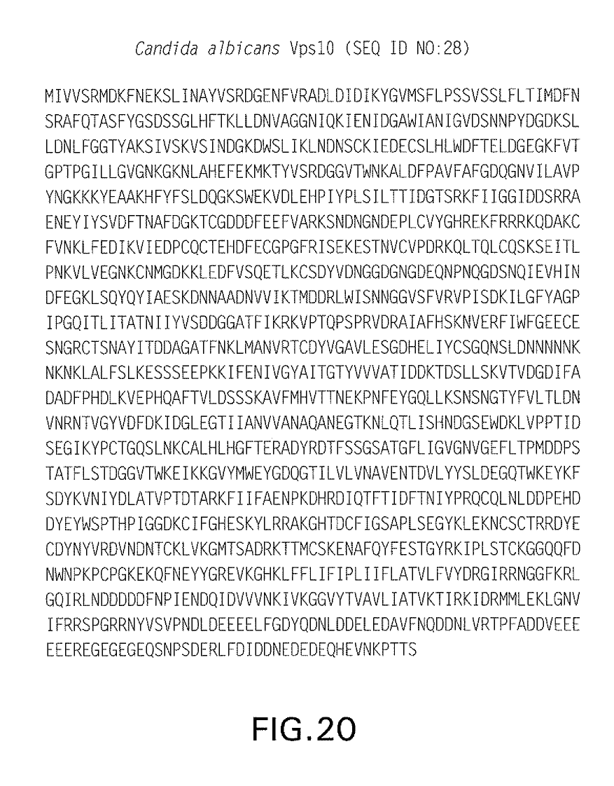

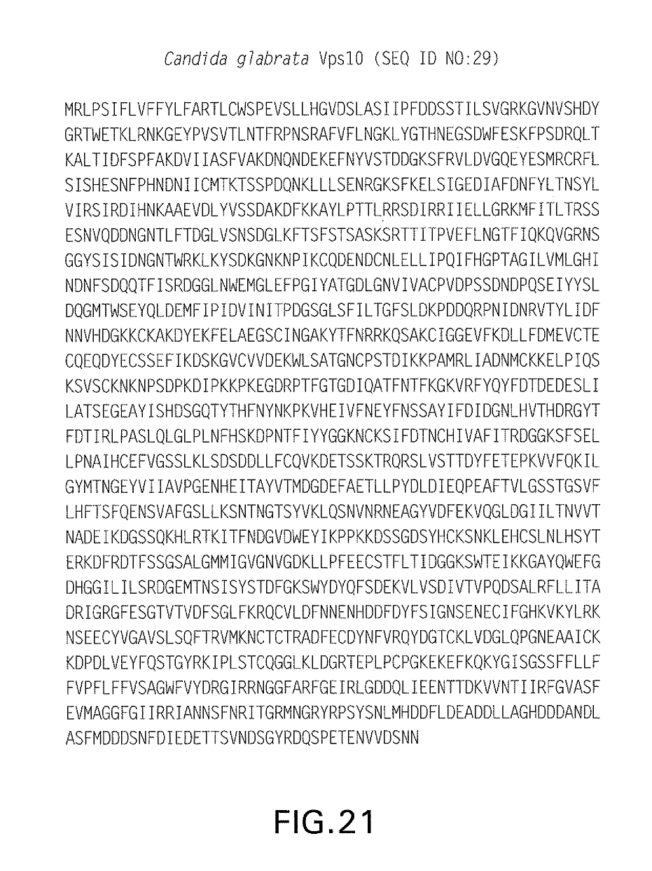

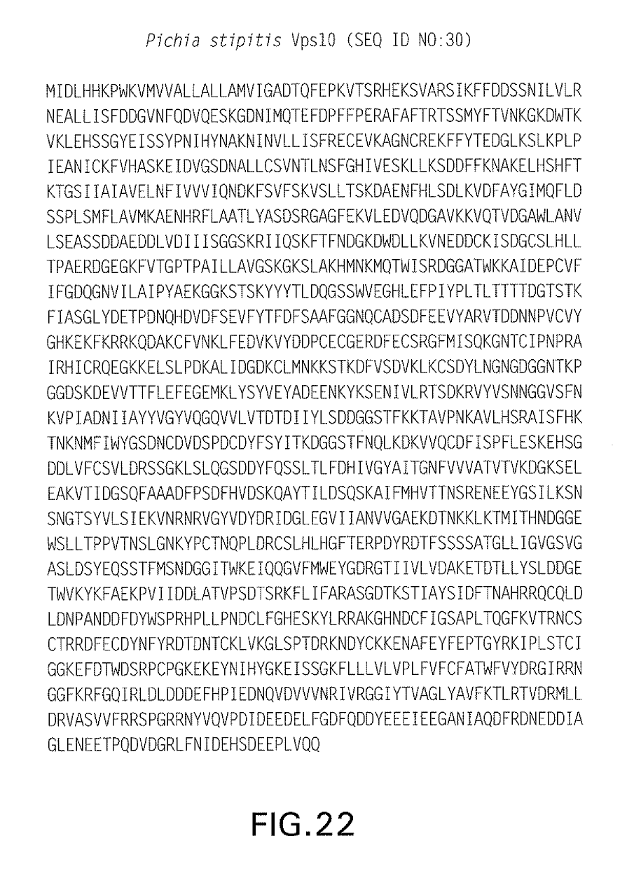

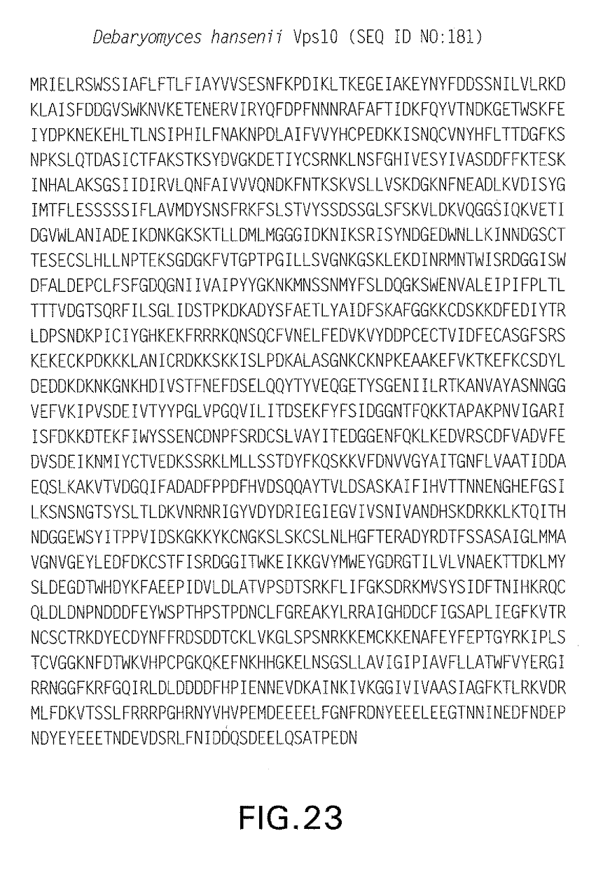

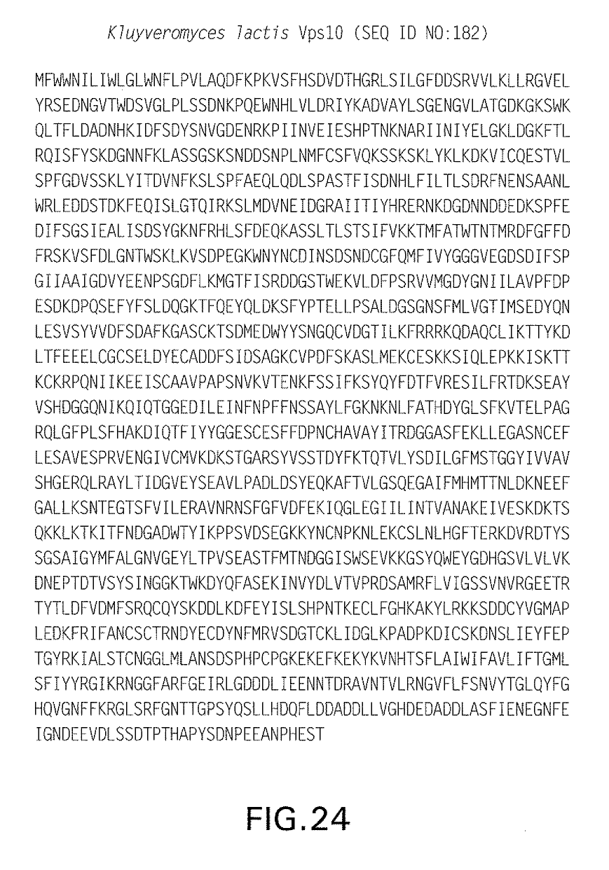

A number of previously known sequences that are Vps 10 homologs are provided herein and are shown in FIGS. 15 and 16 for P. Pastoris ((Vps10-1 and Vps10-2, SEQ ID NOs: 20 and 21, respectively), FIG. 18 for Aspergillus niger (SEQ ID NO:26), FIG. 19 for Saccharomyces pombe (SEQ ID NO:27), FIG. 20 for Candida albicans (SEQ ID NO:28), FIG. 21 for Candida glabrata (SEQ ID NO:29), FIG. 22 for Pichia stipitis (SEQ ID NO:30), FIG. 23 for Debaryomyces hansenii (SEQ ID NO:181), and FIG. 24 for Kluyveromyces lactis (SEQ ID NO:182). Thus, any of these sequences can be targeted for deletion or disruption in the appropriate host cell in order to develop a host cell that lacks vacuolar sorting activity. Use of said host cell in the methods of the present invention, is expected to result in higher levels of recombinant protein production.

Additionally, other genes in S. cerevisiae with homology to Vps10 may perform similar functions and therefore, may be deleted or disrupted in accordance with the invention in order to decrease vacuolar sorting activity and increase heterologous protein yield. For example, S. cerevisiae Vth1p (SEQ ID NO:23), S. cerevisiae Vth2p (SEQ ID NO:24), and S. cerevisiae YNR065c (SEQ ID NO:25)) share homology with Vps10 and are thought to function in a similar manner to Vps10.

Genetic inactivation of VPS10 or a VPS10 gene homolog in the desired host cell can be accomplished by deletion of the Vps 10 open reading frame (ORF) through the use of homologous recombination. Alternatively, the VPS10 gene or a VPS10 gene homolog can also comprise a functional deletion, wherein the complete ORF has not been deleted, but alternate mutations are present that abrogate or disrupt the function of Vps 10, such as partial deletions of the VPS10 gene or homolog, including single codon deletions, point mutations, and substitutions. Other methods that can be used to abrogate the function of Vps10 include, but are not limited to: deletion or disruption of the upstream or downstream regulatory sequences controlling expression of a gene which participates in vacuolar sorting; 2) abrogation or disruption of the vacuolar sorting activity by means of a chemical, peptide, or protein inhibitor; 3) abrogation or disruption of the vacuolar sorting activity by means of nucleic acid-based expression inhibitors, such as antisense RNA, RNA interference, or siRNA; and 4) abrogation or disruption of the vacuolar sorting activity by means of transcription inhibitors or inhibitors of the expression or activity of regulatory factors that control or regulate expression of the gene encoding the enzyme activity.

While methods of increasing the secretion of the recombinant proteins hGCSF and TNFRII-Fc in yeast cells lacking vacuolar sorting activity are shown herein for example, one skilled in the art will recognize that higher levels of any recombinant protein can be achieved through the methods of the present invention, which utilize genetically modified fungal or yeast host cells lacking or comprising reduced vacuolar sorting activity, relative to levels of the recombinant protein produced in wild-type cells. Recombinant proteins comprising an amino acid sequence with homology to the “QRPL” consensus Vps 10 binding sequence can bind to Vps10 in the host cell, leading to alternative trafficking to the vacuole and ultimately reducing protein yield. As discussed in Example 13, van Voorst and colleagues (J Biol Chem 271: 841-6 (1996)) performed mutagenesis of the Cpy “QRPL” peptide near the amino terminus to determine the requirement for sequence conservation to the efficiency of vacuolar sorting. Their analysis revealed that, other than at position Gln²⁴, multiple substitutions could be made without affecting the interaction with Vps10 or leading to missorting. Thus, recombinant proteins do not require absolute homology to the QRPL consensus sequence in order to interact with Vps10 in the host cell, thereby causing a lower yield. Additionally, the S. cerevisiae vacuolar sorting receptor Vps 10 was shown to interact with recombinant proteins, such as E. coli β-lactamase, in an unknown mechanism not involving a “QRPL-like” sorting domain (Holkeri and Makarow, FEBS Lett 429: 162-6 (1998)). Because of the broad potential of recombinant proteins interacting with Vps10 or a Vps10 homolog in the desired host cell, embodiments of the present invention provide broad methods of increasing recombinant yield for a wide range of recombinant proteins, such as therapeutic or biologic protein products through the inactivation or functional deletion of Vps10.

One skilled in the art can easily test for increased protein titers by transforming an expression vector comprising a nucleotide sequence encoding the desired protein into a wild-type yeast or fungal host cell and a host cell of the same species lacking functional Vps10 protein activity and testing for protein expression by, for example, an ELISA assay, a Western blot, a functional activity assay, or any other standard protein detection assay.

In particular aspects of this embodiment of the invention, vacuolar sorting activity is eliminated or reduced from the desired host cell by altering the localization of Vps 10 and/or Vps10 homolog proteins, including P. pastoris Vps10-1, to their site of action in the late Golgi. It is known that in S. cerevisiae, Vps 10 localizes to the late Golgi via protein-protein interactions in the cytoplasmic tail at the carboxy-terminus of the protein (Jorgensen et al., Eur J Biochem 260: 461-9 (1999); Cereghino et al., Mol Biol Cell 6: 1089-102 (1995); Cooper et al., J Cell Biol 133: 529-41, (1996); Dennes et al., J Biol Chem 277: 12288-93 (2002)). Thus, in accordance with the invention, vacuolar sorting activity may be eliminated by single amino acid mutations and/or deletions in the Vps10 cytoplasmic tail, which would alter the localization of Vps10 and prevent sorting of the recombinant protein to the vacuole.

Therefore, this embodiment of the invention relates to methods for producing a recombinant protein in a yeast or fungal host cell comprising: (a) transforming a genetically modified yeast or fungal host cell with an expression vector encoding the protein to produce a host cell, wherein the genetically modified yeast or fungal cell lacks vacuolar sorting activity or has decreased vacuolar sorting activity relative to an unmodified yeast or fungal host cell of the same species, wherein the genetically modified host cell comprises an alteration of the Vps10 cytoplasmic domain that alters its normal trafficking patterns; (b) culturing the transformed host cell in a medium under conditions which induce expression of protein; and (c) isolating the protein from the transformed host cell or culture medium.

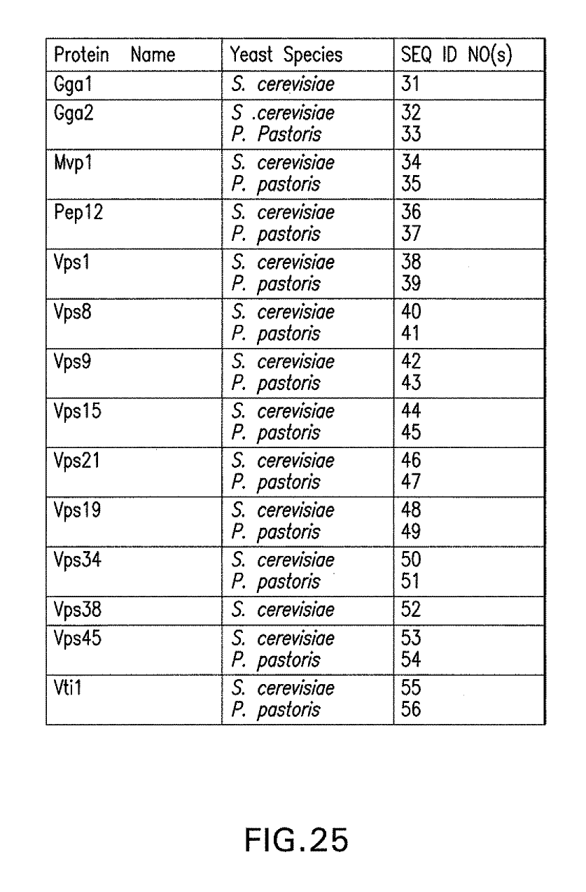

In still other embodiments of the invention, vacuolar sorting activity is reduced or eliminated from the host cell by genetic alterations that functionally delete one or more genes that encode proteins that are associated with the CPY vacuolar sorting pathway, including Gga1, Gga2 (Dell'Angelica et al., J Cell Biol 149: 81-94 (2000)), Mvp1 (Bonangelino et al., Mol Biol Cell 13: 2486-501 (2002)), Pep12 (Robinson et al., Mol Cell Biol 8: 4936-48 (1988)), Vps1, Vps8, Vps9, Vps10, Vps15, Vps21 (Robinson et al., supra), Vps19 (Weisman, L. S. & Wickner, W. J Biol Chem 267: 618-23 (1992)), Vps34 (Schu et al., Science 260: 88-91 (1993)), Vps38 (Rothman et al., Embo J 8: 2057-65 (1989)), Vps45 (Bryant et al., Eur J Cell Biol 76: 43-52 (1998)), and Vti1 (von Mollard et al., J Cell Biol 137: 1511-24 (1997)). Amino acid sequences of proteins associated with the CPY vacuolar sorting pathway are provided herein (see FIG. 25).

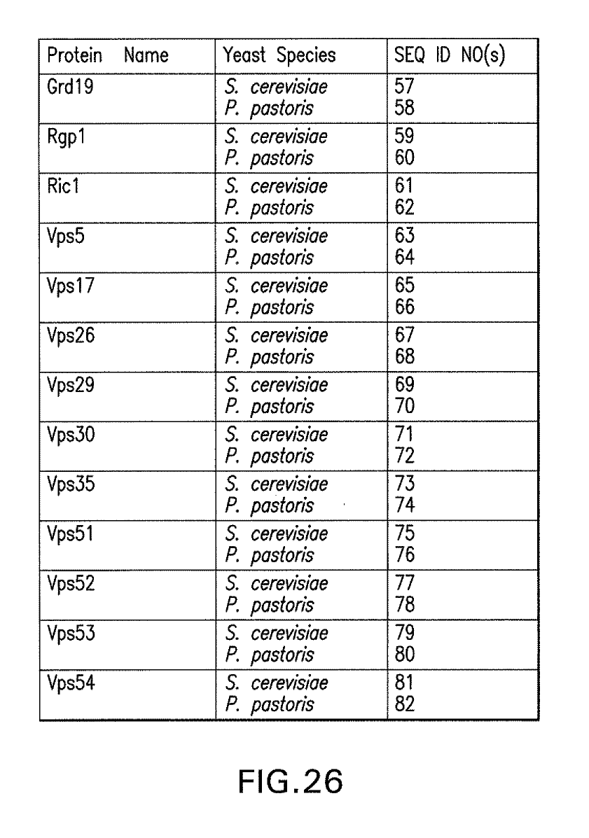

In further embodiments of the invention, vacuolar sorting activity is reduced or eliminated from the host cell by genetic alterations that functionally delete one or more genes that encode proteins that are associated with the recycling of Vps 10 to the late Golgi from the PVC (Seaman et al., J Cell Biol 137: 79-92, (1997); Mullins et al. Bioessays 23: 333-43 (2001)), including Grd19 (Hettema et al. Embo J 22: 548-57 (2003)), Rgp1, Ric1 (Bonangelino et al. Mol Biol Cell 13: 2486-501 (2002)), Vps5, Vps17, Vps26 (Robinson et al., Mol Cell Biol 8: 4936-48 (1988)), Vps29 (Rothman et al., Embo J 8: 2057-65 (1989)), Vps30, Vps35 (Robinson et al., supra), Vps51 (Conibear et al., Mol Biol Cell 14: 1610-23 (2003)), Vps52, Vps53 and Vps54 (Conibear et al., Mol Biol Cell 11: 305-23 (2000)). Amino acid sequences of proteins associated with the recycling of Vps 10 are provided herein (see FIG. 26).

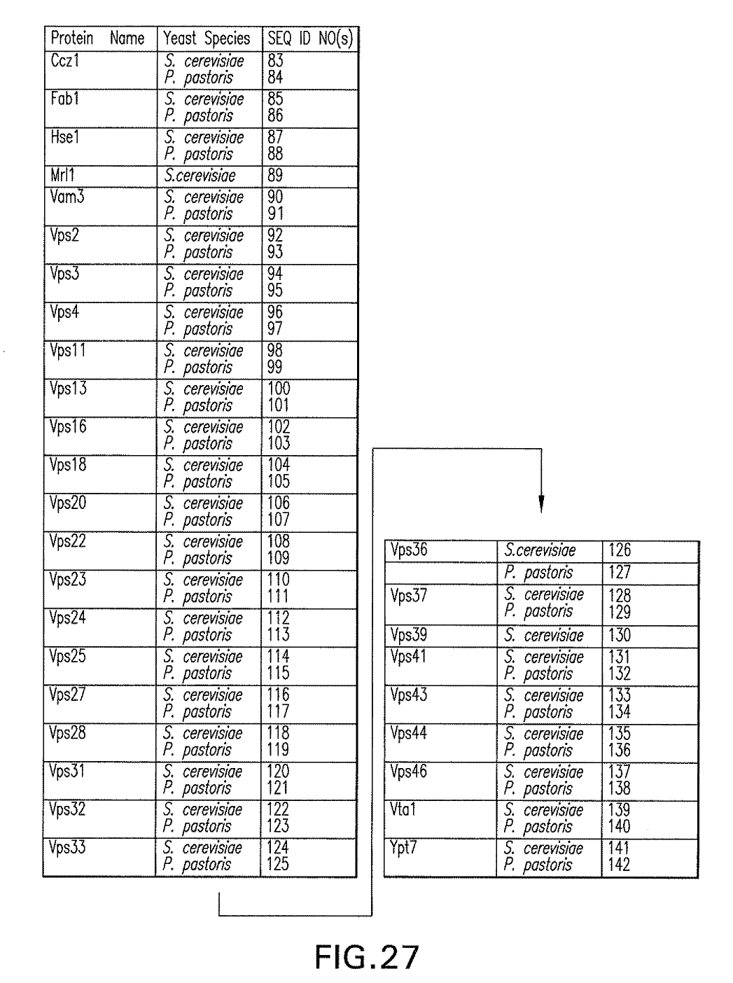

In still further embodiments, vacuolar sorting activity is reduced or eliminated from the host cell by genetic alterations that functionally delete genes that encode proteins associated with proper MVB function and/or fusion to the vacuole, including: Ccz1 (Kucharczyk et al., J Cell Sci 113 Pt 23: 4301-11 (2000)), Fab1 (Yamamoto et al., Mol Biol Cell 6: 525-39 (1995)), Hse1 (Bilodeau et al., J Cell Biol 163: 237-43 (2003)), Mrl1 (Bonangelino et al., Mol Biol Cell 13: 2486-501 (2002)), Vam3 (Nichols et al., Nature 387: 199-202 (1997)), Vps2, Vps3, Vps4 (Robinson et al., supra), Vps11 (Rothman et al., supra), Vps13, Vps16, Vps18 (Robinson et al., supra), Vps20 (Yeo et al., J Cell Sci 116: 3957-70 (2003)), Vps22, Vps23, Vps24, Vps25, Vps27, Vps28, Vps31, Vps32, Vps33, Vps36 (Robinson et al., supra), Vps37, Vps39 (Rothman et al., supra), Vps41 (Nakamura et al., J Biol Chem 272: 11344-9 (1997)), Vps43 (Sato et al., Mol Cell Biol 18: 5308-19 (1998)), Vps44 (Bowers et al., Mol Biol Cell 11: 4277-94 (2000)), Vps46 (Amerik et al., Mol Biol Cell 11: 3365-80 (2000)), Vta1 (Yeo et al., supra), and Ypt7 (Tsukada et al., J Cell Sci 109 (Pt 10): 2471-81 (1996)). Amino acid sequences of proteins associated with proper MVB function and/or fusion to the vacuole are provided herein (see FIG. 27).

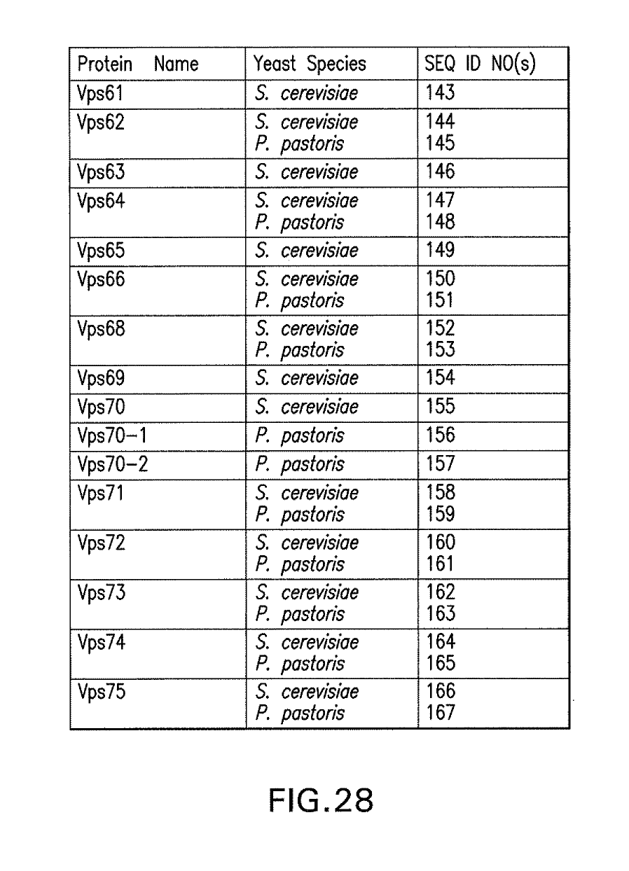

In alternative embodiments of the methods described herein, vacuolar sorting activity is reduced or eliminated from the host cell by genetic alterations that functionally delete one or more genes that encode proteins required for proper Cpy vacuolar targeting through unknown mechanisms, including: Vps61, Vps62, Vps63, Vps64, Vps65, Vps66, Vps68, Vps69, Vps70, Vps71, Vps72, Vps73, Vps74, and Vps75 (Bonangelino et al., Mol Biol Cell 13: 2486-501 (2002)). Amino acid sequences of proteins associated with proper Cpy vacuolar targeting through unknown mechanisms are provided herein (see FIG. 28).

The invention also relates to methods for increasing the yield of heterologous proteins produced in yeast cells by eliminating or reducing vacuolar sorting activity, wherein vacuolar sorting activity is abrogated or disrupted by means of a chemical, peptide, or protein inhibitor. In this aspect of the invention, a peptide inhibitor can be utilized that blocks Vps10, Vps10-1 or other homolog of Vps10, for example, a peptide of Pro-Cpy can be expressed while expressing the heterologous protein of interest. The Pro-Cpy peptides will bind to and saturate Vps10-1, thereby preventing binding of the heterologous protein. Chemical inhibitors are also useful for abrogating vacuolar sorting activity. In preferred embodiments of this aspect of the invention, the chemical inhibitor is a small chemical inhibitor referred to as a sortie. It is known that sortins interfere with the vacuolar delivery of proteins in plants and yeast (Norambuena et al., BMC Chem Biol 8: 1 (2008); Zouhar et al. Proc Natl Acad Sci USA 101: 9497-501 (2004)). In accordance with the invention, sortins are added to the cell culture, for example, during yeast fermentation, thereby increasing yield of the heterologous protein of interest through elimination of vacuolar sorting and degradation. One skilled in the art will realize that the sortins should then be cleared from the purified recombinant protein when using this method for therapeutic protein production.

The invention further relates to a method of increasing the yield of heterologous protein production, wherein the heterologous protein comprises a Vps10 binding site, comprising introducing a modification to the amino acid sequence of the heterologous protein which prevents binding of the protein to S. cerevisiae Vps 10 or a Vps 10 homolog such as P. pastoris Vps10-1. As described in Example 13, recombinant proteins which comprise a “QRPL-like” sorting signal would likely bind to Vps10 if the sorting peptide was surface exposed and direct the recombinant protein to the yeast vacuole. Previous methods for eliminating vacuolar sorting activity, described supra, include methods that target Vps 10 through genetic inactivation of a gene that encodes Vps10 or a Vps10 homolog. In the alternative embodiment described here, the recombinant protein or gene encoding the recombinant protein itself is mutated to prevent binding to Vps 10 or a Vps 10 homolog such as Vps 10-1. Consistent with the paper by van Voorst et al. (J. Biol. Chem. 271:841-6 (1996), the Gln residue of the Gln-Arg-Pro-Leu (SEQ ID NI:176) Vps10 sorting signal is targeted for disruption in this embodiment of the invention because this residue is required for Vps10 interaction.

Thus, the invention also relates to a modified recombinant protein comprising a “QRPL-like” sorting signal, wherein the Q residue of the “QRPL-like” sorting signal is modified, either by deletion or substitution.

In other aspects, the invention relates to methods of producing higher levels of a modified recombinant protein comprising a QRPL-like sorting signal relative to the unmodified protein; the method comprising (1) expressing a modified nucleotide sequence encoding the protein in a yeast or fungal host cell in culture medium under conditions which favor expression of the protein; wherein the nucleotide sequence is mutated such that the QRPL-like sorting signal of the recombinant protein is rendered nonfunctional; and (2) isolating the protein from the host cell or culture medium.

Any fungal or yeast strain can be used as the basis for developing a genetically modified host cell for use in the methods of the present invention. Said genetically modified host cell is modified by inactivating vacuolar sorting activity, for example, by functionally deleting Vps 10 or a Vps 10 homolog, such as by deleting or disrupting a gene encoding the Vps 10 or Vps 10 protein homolog.

Yeast host cells useful in the methods of the present invention include, but are not limited to: Pichia pastoris, Saccharomyces cerevisiae, Saccharomyces pombe, Candida albicans, Candida glabrata, Pichia stipitis, Hansenula polymorpha, Kluyvermyces fragilis, Kluyveromyces sp., Kluveromyces lactis, Schizosaccharomyces pombe, Pichia finlandica, Pichia trehalophila, Pichia koclamae, Pichia thermotolerans, Pichia salictaria, Pichia minuta (Ogataea minuta, Pichia lindneri), Pichia guercuum, Pichia pijperi, Pichia sp., Saccharomyces sp., Pichia membranaefaciens, Pichia opuntiae, and Pichia methanolica.

Additional fungal host cells useful in the methods described herein include Aspergillus nidulans, Aspergillus niger, Aspergillus oryzae, Trichoderma reesei, Chrysosporium lucknowense, Fusarium sp., Fusarium gramineum, Fusarium venenatum, and Neurospora crassa.

In preferred embodiments of the methods described herein, the yeast or fungal host cell is selected from the group consisting of: Pichia pastoris, Saccharomyces cerevisiae, Aspergillus niger, Saccharomyces pombe, Candida albicans, Candida glabrata, Pichia stipitis, Debaryomyces hansenii, Kluyveromyces lactis, and Hansenula polymorpha. In further preferred embodiments, the host cell is a Pichia cell. In some preferred embodiments, the host cell is Pichia pastoris or Saccharomyces cerevisiae. In specific embodiments, the host cell is Pichia pastoris.

In other aspects, the invention relates to a modified fungal host cell which comprises a functional deletion or knock-out of Vps10 activity, wherein the host cell comprises an expression vector comprising a sequence of nucleotides that encodes a heterologous protein.

In a particular embodiment, the invention relates to a Pichia pastoris cell lacking vacuolar sorting activity or having reduced vacuolar sorting activity relative to a wild-type Pichia pastoris cell, wherein the host cell comprises a functional deletion of a Vps10-1 protein, for example, the Vps10-1 set forth in SEQ ID NO:20. The Pichia pastoris cell may be further modified by transforming the cell with an expression vector that comprises a sequence of nucleotides that encodes a heterologous protein, such as a biologic or therapeutic protein, to produce a modified host cell. Said cells are useful to produce high titers of the heterologous protein by increasing its secretion efficiency. In preferable embodiments of this aspect of the invention, the host cell comprises a VPS10-2 gene, for example the VPS10-2 set forth in SEQ ID NO:21 that is not deleted.

In further embodiments of the invention, the heterologous protein produced in the host cell is a glycoprotein. In said embodiments, it may be useful to further modify the host cell in order to produce a glycoprotein in which the glycosylation pattern is human-like, as described, infra.

The modified yeast host cells of the present invention, which lack vacuolar sorting activity or have reduced vacuolar sorting activity relative to an unmodified yeast cell of the same species, may be further modified to express glycoproteins in which the glycosylation pattern is human-like or humanized. Modifying the yeast host cell in this manner can be achieved by eliminating selected endogenous glycosylation enzymes and/or supplying exogenous enzymes as described by for example, Gerngross, U.S. Pat. No. 7,029,872 and Gerngross et al., U.S. Published Application No. 20040018590. For example, a host cell can be selected or engineered to be depleted in 1,6-mannosyl transferase activities (e.g., ΔOCH1), which would otherwise add mannose residues onto the N-glycan on a glycoprotein.

In one embodiment, the host cell further includes an α1,2-mannosidase catalytic domain fused to a cellular targeting signal peptide not normally associated with the catalytic domain and selected to target the α1,2-mannosidase activity to the ER or Golgi apparatus of the host cell where it can operate optimally. These host cells produce glycoproteins comprising a Man₅GlcNAc₂ glycoform. For example, U.S. Pat. No. 7,029,872 and U.S. Published Patent Application Nos. 2004/0018590 and 2005/0170452 disclose lower eukaryote host cells capable of producing a glycoprotein comprising a Man₅GlcNAc₂ glycoform.

In a further embodiment, the host cell further includes a GlcNAc transferase I (GnT I) catalytic domain fused to a cellular targeting signal peptide not normally associated with the catalytic domain and selected to target GlcNAc transferase I activity to the ER or Golgi apparatus of the host cell where it can operate optimally. These host cells produce glycoproteins comprising a GlcNAcMan₅GlcNAc₂ glycoform. U.S. Pat. No. 7,029,872 and U.S. Published Patent Application Nos. 2004/0018590 and 2005/0170452 disclose lower eukaryote host cells capable of producing a glycoprotein comprising a GlcNAcMan₅GlcNAc₂ glycoform.

In yet another embodiment, the host cell further includes a mannosidase II catalytic domain fused to a cellular targeting signal peptide not normally associated with the catalytic domain and selected to target mannosidase II activity to the ER or Golgi apparatus of the host cell where it can operate optimally. These host cells produce glycoproteins comprising a GlcNAcMan₃GlcNAc₂ glycoform. U.S. Pat. No. 7,029,872 and U.S. Published Patent Application No. 2004/0230042 discloses lower eukaryote host cells that express mannosidase II enzymes and are capable of producing glycoproteins having predominantly a GlcNAc₂Man₃GlcNAc₂ glycoform.

In a further embodiment, the host cell further includes GlcNAc transferase II (GnT II) catalytic domain fused to a cellular targeting signal peptide not normally associated with the catalytic domain and selected to target GlcNAc transferase II activity to the ER or Golgi apparatus of the host cell where it can operate optimally. These host cells produce glycoproteins comprising a GlcNAc₂Man₃GlcNAc₂ glycoform. U.S. Pat. No. 7,029,872 and U.S. Published Patent Application Nos. 2004/0018590 and 2005/0170452 disclose lower eukaryote host cells capable of producing glycoproteins comprising a GlcNAc₂Man₃GlcNAc₂ glycoform.

In a further embodiment, the host cell further includes a galactosyltransferase catalytic domain fused to a cellular targeting signal peptide not normally associated with the catalytic domain and selected to target galactosyltransferase activity to the ER or Golgi apparatus of the host cell where it can operate optimally. These host cells produce glycoproteins comprising a GalGlcNAc₂Man₃GlcNAc₂ or Gal₂GlcNAc₂Man₃GlcNAc₂ glycoform, or mixture thereof. U.S. Pat. No. 7,029,872 and U.S. Published Patent Application No. 2006/0040353 discloses lower eukaryote host cells capable of producing glycoproteins comprising a Gal₂GlcNAc₂Man₃GlcNAc₂ glycoform.

In a further embodiment, the host cell further includes a sialyltransferase catalytic domain fused to a cellular targeting signal peptide not normally associated with the catalytic domain and selected to target sialytransferase activity to the ER or Golgi apparatus of the host cell. These host cells produce glycoproteins comprising predominantly a NANA₂Gal₂GlcNAc₂Man₃GlcNAc₂ glycoform or NANAGal₂GlcNAc₂Man₃GlcNAc₂ glycoform or mixture thereof. It is useful that the host cell further include a means for providing CMP-sialic acid for transfer to the N-glycan. U.S. Published Patent Application No. 2005/0260729 discloses a method for genetically engineering lower eukaryotes to have a CMP-sialic acid synthesis pathway and U.S. Published Patent Application No. 2006/0286637 discloses a method for genetically engineering lower eukaryotes to produce sialylated glycoproteins.

Any one of the preceding host cells can further include one or more GlcNAc transferase selected from the group consisting of GnT III, GnT IV, GnT V, GnT VI, and GnT IX to produce glycoproteins having bisected (GnT III) and/or multiantennary (GnT IV, V, VI, and IX) N-glycan structures such as disclosed in U.S. Published Patent Application Nos. 2004/074458 and 2007/0037248.

In still further embodiments, the host cell that produces glycoproteins that have predominantly GlcNAcMan₅GlcNAc₂ N-glycans further includes a galactosyltransferase catalytic domain fused to a cellular targeting signal peptide not normally associated with the catalytic domain and selected to target Galactosyltransferase activity to the ER or Golgi apparatus of the host cell. These host cells produce glycoproteins comprising predominantly the GalGlcNAcMan₅GlcNAc₂ glycoform.

In a further embodiment, the host cell that produced glycoproteins that have predominantly the GalGlcNAcMan₅GleNAc₂ N-glycans further includes a sialyltransferase catalytic domain fused to a cellular targeting signal peptide not normally associated with the catalytic domain and selected to target sialytransferase activity to the ER or Golgi apparatus of the host cell. These host cells produce glycoproteins comprising a NANAGalGlcNAcMan₅GlcNAc₂ glycoform.

Various of the preceding host cells further include one or more sugar transporters such as UDP-GlcNAc transporters (for example, Kluyveromyces lactis and Mus musculus UDP-GlcNAc transporters), UDP-galactose transporters (for example, Drosophila melanogaster UDP-galactose transporter), and CMP-sialic acid transporter (for example, human sialic acid transporter). Because Pichia pastoris lacks the above transporters, it is preferable that the Pichia pastoris be genetically engineered to include the above transporters.

To reduce or eliminate detectable cross reactivity to antibodies against host cell protein, the recombinant glycoengineered yeast host cells can be genetically engineered to eliminate glycoproteins having α-mannosidase-resistant N-glycans by deleting or disrupting one or more of the β-mannosyltransferase genes (e.g., BMT1, BMT2, BMT3, and BMT4) (See, U.S. Published Patent Application No. 2006/0211085) and glycoproteins having phosphomannose residues by deleting or disrupting one or both of the phosphomannosyl transferase genes PNO1 and MNN4B (See for example, U.S. Pat. Nos. 7,198,921 and 7,259,007), which in further aspects can also include deleting or disrupting the MNN4A gene. Disruption includes disrupting the open reading frame encoding the particular enzymes or disrupting expression of the open reading frame or abrogating translation of RNAs encoding one or more of the β-mannosyltransferases and/or phosphomannosyltransferases using interfering RNA, antisense RNA, or the like. The host cells can further include any one of the aforementioned host cells modified to produce particular N-glycan structures.

Regulatory sequences which may be used in the practice of the methods disclosed herein include signal sequences, promoters, and transcription terminator sequences. Examples of promoters include promoters from numerous species, including but not limited to alcohol-regulated promoter, tetracycline-regulated promoters, steroid-regulated promoters (e.g., glucocorticoid, estrogen, ecdysone, retinoid, thyroid), metal-regulated promoters, pathogen-regulated promoters, temperature-regulated promoters, and light-regulated promoters. Specific examples of regulatable promoter systems well known in the art include but are not limited to metal-inducible promoter systems (e.g., the yeast copper-metallothionein promoter), plant herbicide safner-activated promoter systems, plant heat-inducible promoter systems, plant and mammalian steroid-inducible promoter systems, Cym repressor-promoter system (Krackeler Scientific, Inc. Albany, N.Y.), RheoSwitch System (New England Biolabs, Beverly Mass.), benzoate-inducible promoter systems (See WO2004/043885), and retroviral-inducible promoter systems. Other specific regulatable promoter systems well-known in the art include the tetracycline-regulatable systems (See for example, Berens & Hillen, Eur Biochem 270: 3109-3121 (2003)), RU 486-inducible systems, ecdysone-inducible systems, and kanamycin-regulatable system. Lower eukaryote-specific promoters include but are not limited to the Saccharomyces cerevisiae TEF-1 promoter, Pichia pastoris GAPDH promoter, Pichia pastoris GUT1 promoter, PMA-1 promoter, Pichia pastoris PCK-1 promoter, and Pichia pastoris AOX-1 and AOX-2 promoters.

Examples of transcription terminator sequences include transcription terminators from numerous species and proteins, including but not limited to the Saccharomyces cerevisiae cytochrome C terminator; and Pichia pastoris ALG3 and PMA1 terminators.

Yeast selectable markers include drug resistance markers and genetic functions which allow the yeast host cell to synthesize essential cellular nutrients, e.g. amino acids. Drug resistance markers which are commonly used in yeast include chloramphenicol, kanamycin, methotrexate, G418 (geneticin), Zeocin, and the like. Genetic functions which allow the yeast host cell to synthesize essential cellular nutrients are used with available yeast strains having auxotrophic mutations in the corresponding genomic function. Common yeast selectable markers provide genetic functions for synthesizing leucine (LEU2), tryptophan (TRP1 and TRP2), praline (PRO1), uracil (URA3, URA5, URA6), histidine (HIS3), lysine (LYS2), adenine (ADE1 or ADE2), and the like. Other yeast selectable markers include the ARR3 gene from S. cerevisiae, which confers arsenite resistance to yeast cells that are grown in the presence of arsenite (Bobrowicz et al., Yeast, 13:819-828 (1997); Wysocki et al., J. Biol. Chem. 272:30061-30066 (1997)).

A number of suitable integration sites include those enumerated in U.S. Published application No. 2007/0072262 and include homologs to loci known for Saccharomyces cerevisiae and other yeast or fungi. Methods for integrating vectors into yeast are well known, for example, See U.S. Pat. No. 7,479,389, PCT Published Application No. WO2007136865, and PCT/US2008/13719. Examples of insertion sites include, but are not limited to, Pichia ADE genes; Pichia TRP (including TRP1 through TRP2) genes; Pichia MCA genes; Pichia CYM genes; Pichia PEP genes; Pichia PRB genes; and Pichia LEU genes. The Pichia ADE1 and ARG4 genes have been described in Lin Cereghino et al., Gene 263:159-169 (2001) and U.S. Pat. No. 4,818,700, the HIS3 and TRP1 genes have been described in Cosano et al., Yeast 14:861-867 (1998), HIS4 has been described in GenBank Accession No. X56180.

All publications mentioned herein are incorporated by reference for the purpose of describing and disclosing methodologies and materials that might be used in connection with the present invention. Nothing herein is to be construed as an admission that the invention is not entitled to antedate such disclosure by virtue of prior invention.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defined in the appended claims.

The following examples illustrate, but do not limit the invention.

### Materials and Methods:

### Example 1

### Strains and Media.

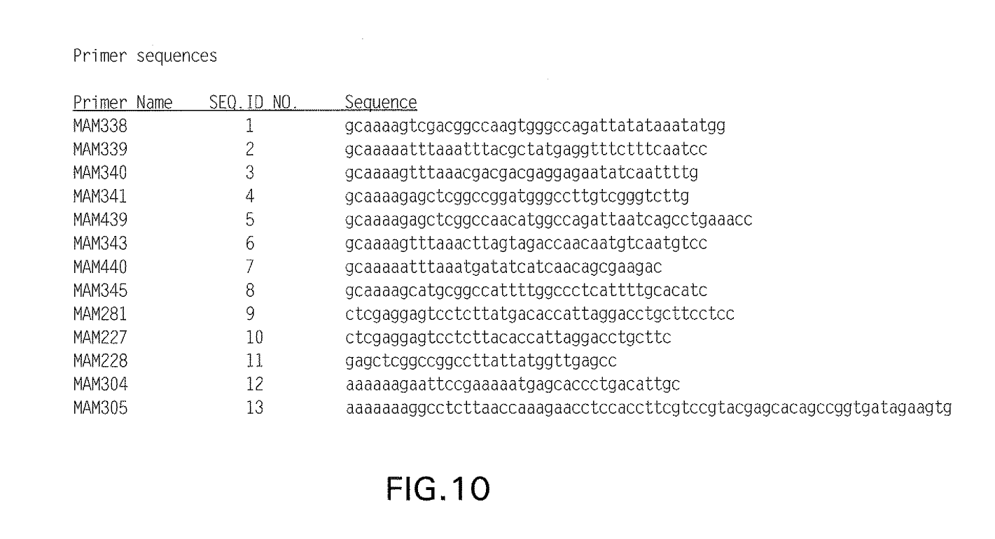

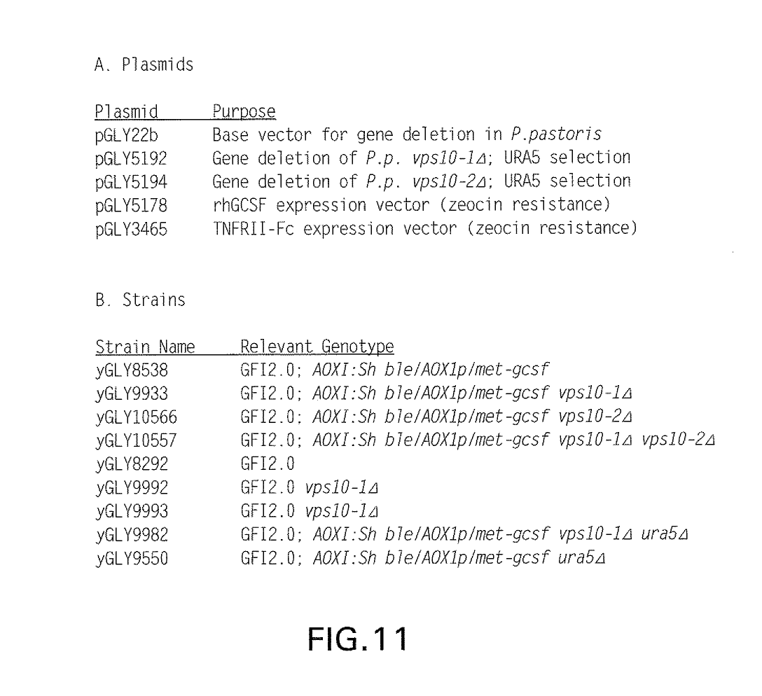

K coli strain TOP10 was used for recombinant DNA work. All primers and plasmids and selected Pichia pastoris strains used in this study are listed in FIGS. 10 and 11. Protein expression was carried out with buffered glycerol-complex medium (BMGY) and buffered methanol-complex medium (BMMY). BMGY medium consisted of 2% martone, 100 mM potassium phosphate buffer at pH 6.0, 1.34% yeast nitrogen base, 0.00002% biotin, and 2% glycerol as a growth medium. BMMY contained the same components as BMGY, except 1% methanol was used as an induction medium instead of glycerol. YMD medium consisted of 2% martone, 2% dextrose and 2% agar and was used to grow Pichia pastoris strains on agar plates. Restriction and modification enzymes were purchased from New England BioLabs (Beverly, Mass.). Oligonucleotides were obtained from Integrated DNA Technologies (Coralville, Iowa). Salts and buffering agents were obtained from Sigma (St. Louis, Mo.).

### Example 2

### Transformation of Yeast Strains.

Yeast transformations with expression/integration vectors were as discussed, infra (Cregg et al., Mol. Biotechnol. 16: 23-52 (2000)). Pichia pastoris strains were grown in 50 mL YMD media overnight to an OD ranging from 0.2 to 6.0. After incubation on ice for 30 minutes, cells were pelleted by centrifugation at 2500-3000 rpm for 5 minutes. The media was removed and the cells were washed three times with ice cold sterile 1M sorbitol. The cell pellet was then resuspended in 0.5 ml ice cold sterile 1M sorbitol. Ten 4 linearized DNA (1-10 μg) and 100 μL cell suspension were combined in an electroporation cuvette and incubated for 5 minutes on ice. Electroporation was performed using a Bio-Rad GenePulser Xcell (Bio-Rad Laboratories, Hercules, Calif.), following a preset Pichia pastoris protocol (2 kV, 25 μF, 200Ω). Immediately following electroporation, 1 mL YMDS recovery media (YMD media plus 1 M sorbitol) was added to the mixture. The transformed cells were allowed to recover for a length of time ranging from four hours to overnight at room temperature (26° C.). After cell recovery, the cells were plated on selective media.

### Example 3

Identification of Vps10 Homologs in P. pastoris.

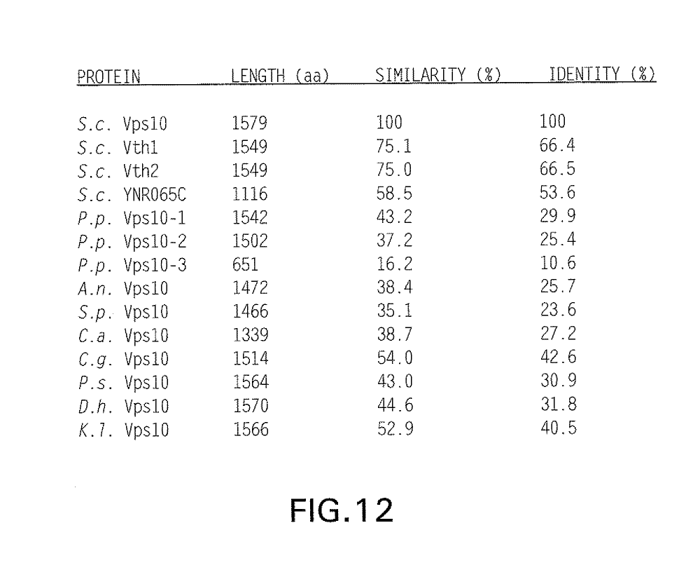

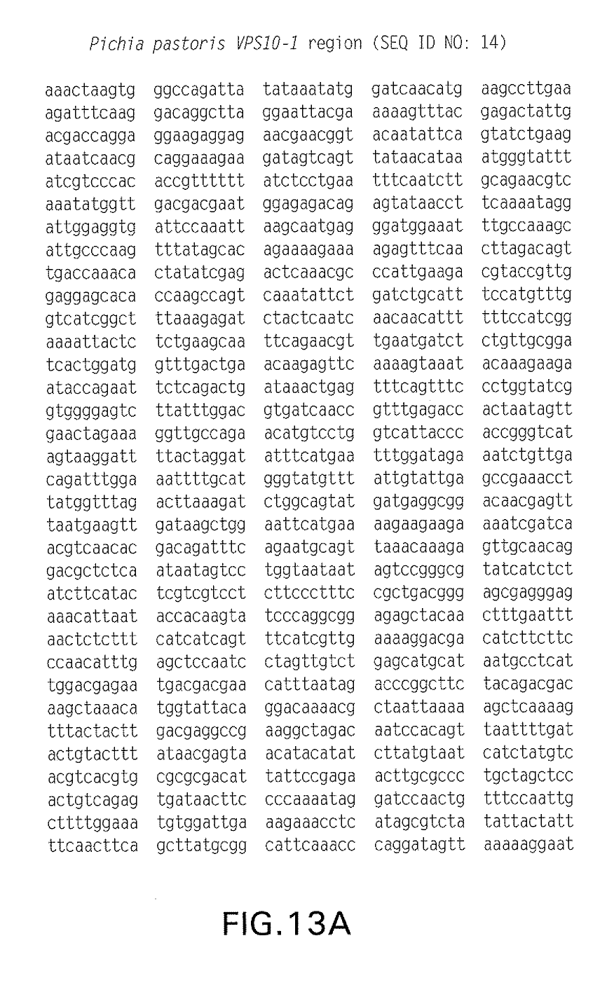

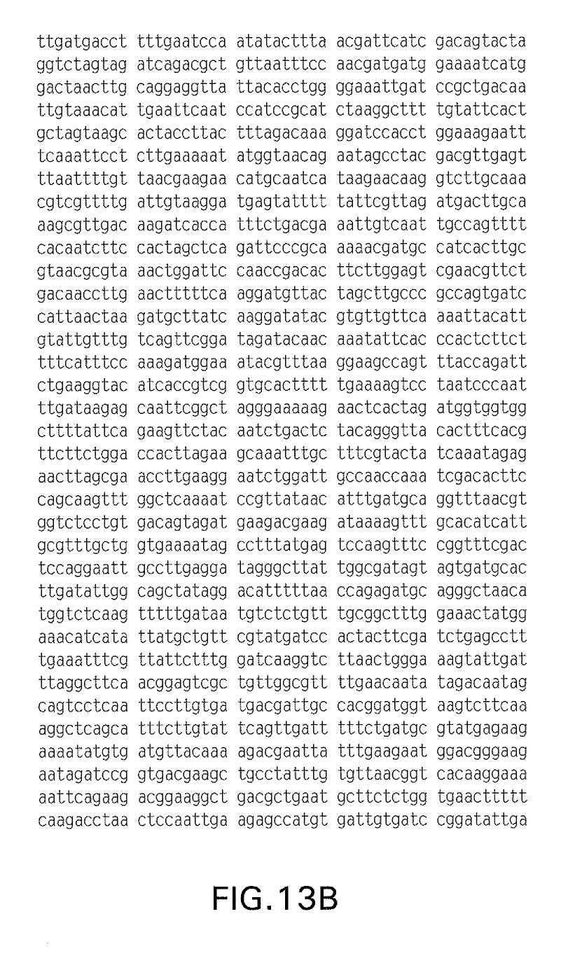

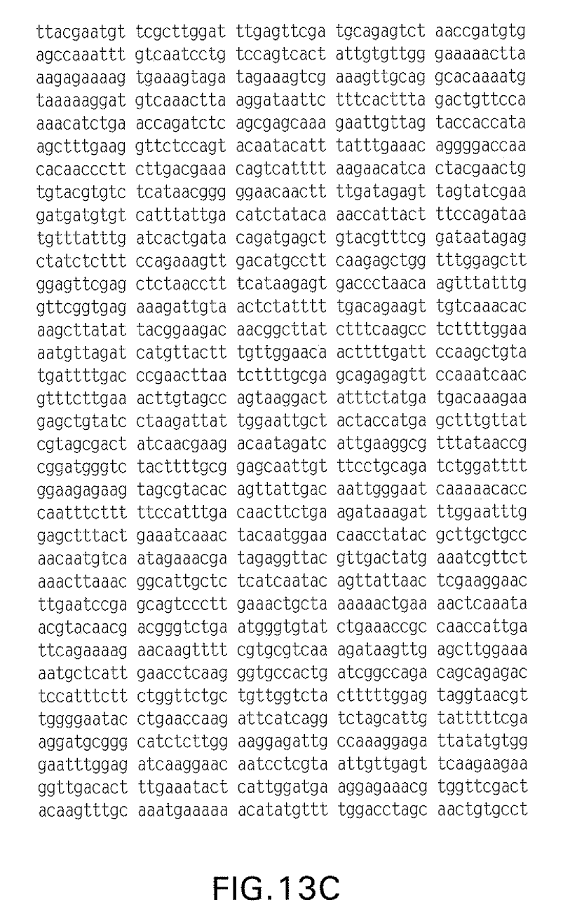

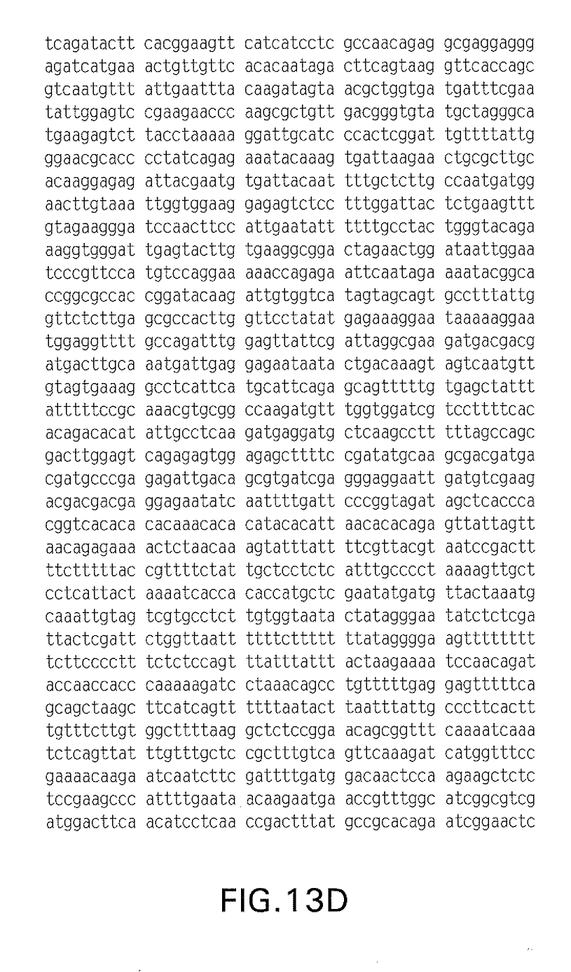

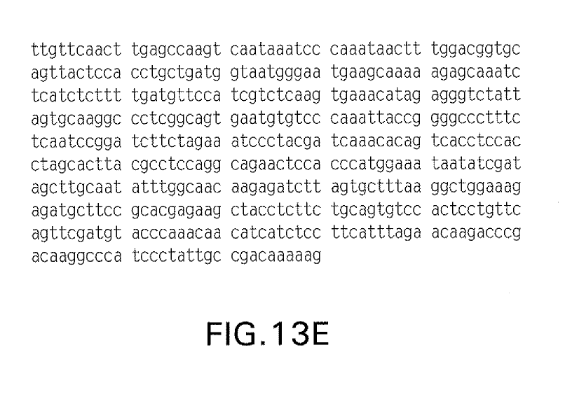

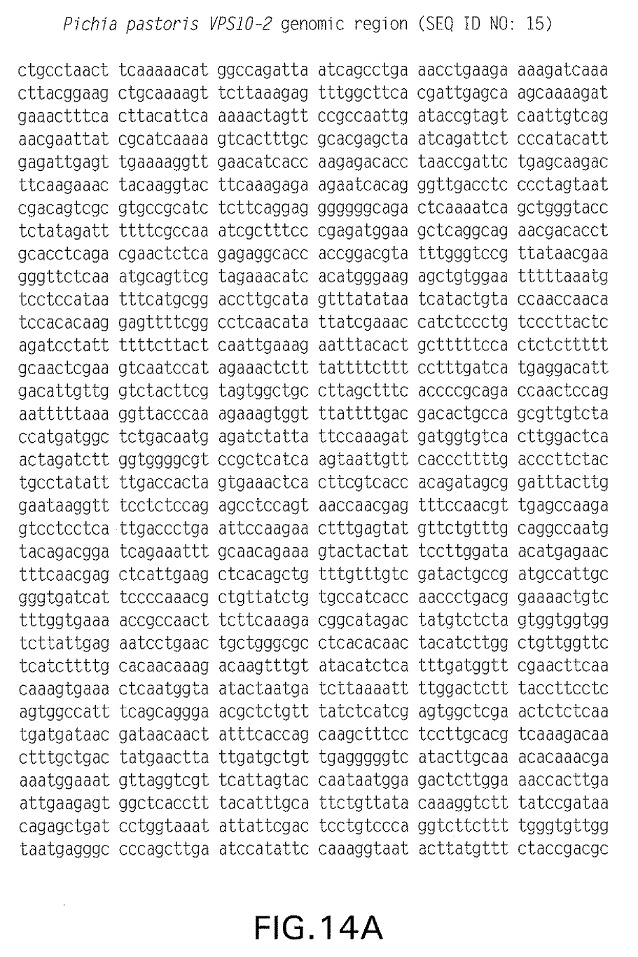

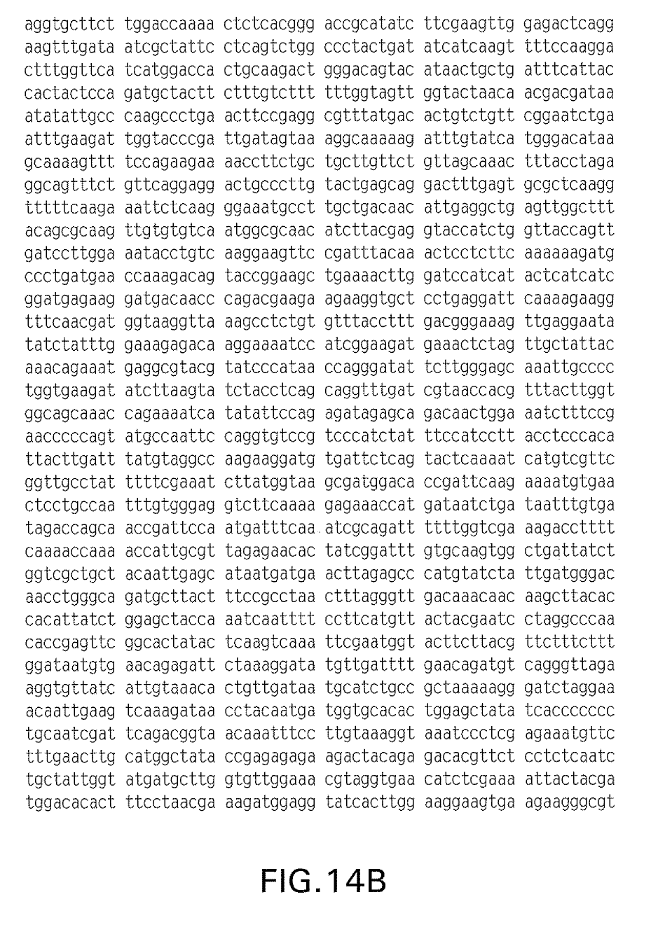

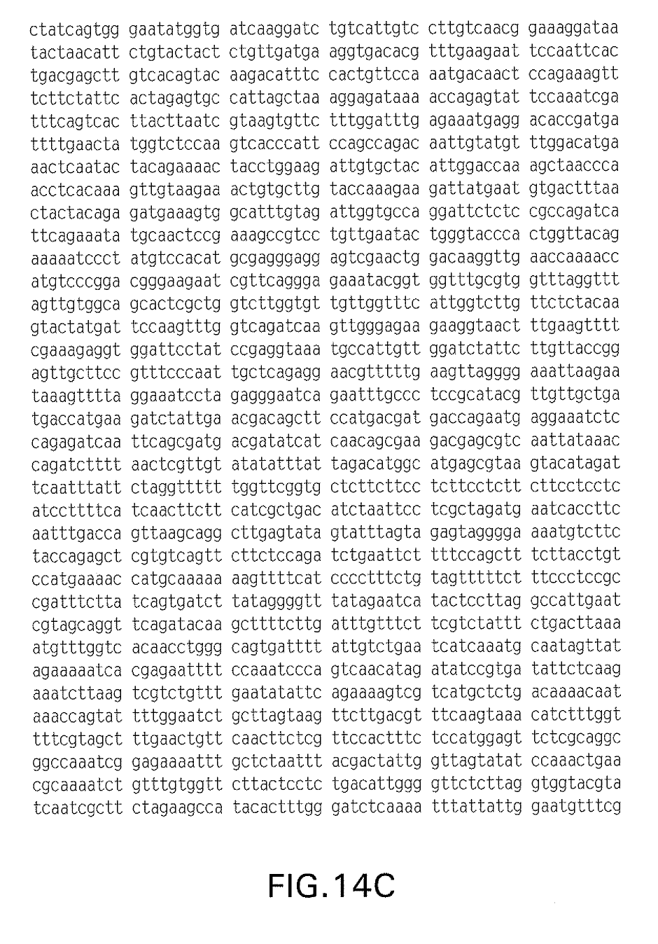

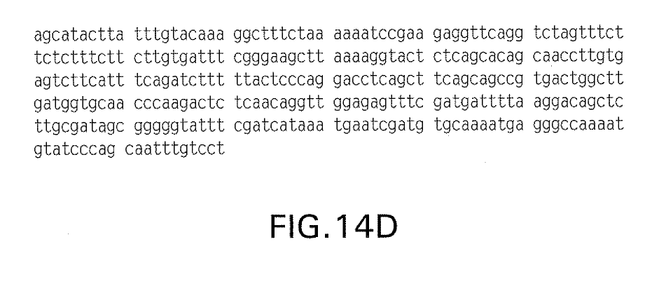

Protein sequences of the four Vps10 homologs (Vps10p/Pep1p/Vpt1p (SEQ ID NO:22), Vth1p (SEQ ID NO:23), Vth2p (SEQ ID NO:24), and YNR065c (SEQ ID NO:25)) in S. cerevisiae were obtained from Genbank®. As discussed in Example 14, potential VPS10 gene homologs were identified in Pichia pastoris using the four S. cerevisiae proteins (above) in a TBLASTN computational search (Altschul et al., J. Mol. Biol. 215(3): 403-10 (1990); Altschul et al., Nucleic Acids Res. 25:3389-3402 (1997)) of a proprietary Pichia pastoris genome. Two Pichia gene homologs, named VPS10-1 and VPS10-2, were identified. Genomic DNA sequences for VPS10-1 (SEQ ID NO:14) and VPS10-2 (SEQ ID NO:15) are provided in FIGS. 13 and 14, respectively. Translated protein sequences for Vps10-1p (SEQ ID NO:20) and Vps10-2p (SEQ ID NO:21) are provided in FIGS. 15 and 16, respectively. A comparison of the amino acid sequences of the P. pastoris Vps10p homologs to S. cerevisiae Vps10p, as well as to other fungal strains, is shown in FIG. 12.

### Example 4

### Generation of Gene Deletion Plasmids.

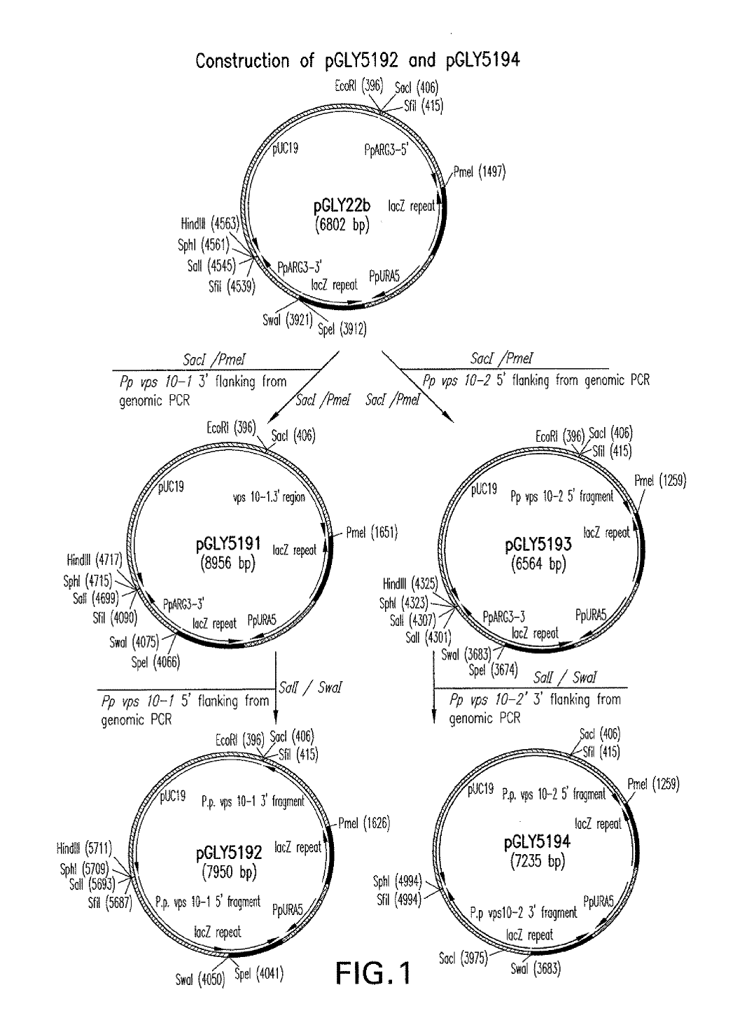

The plasmid pGLY5192 was constructed to delete the open reading frame of the VPS10-1 gene (see FIG. 1) and create a yeast strain deficient in vacuolar sorting receptor (Vps10-1p) activity. To generate the vps10-1Δ knock-out plasmid pGLY5192, the upstream 5′ flanking region was first amplified using routine PCR conditions with primers MAM338 (SEQ ID NO:1) and MAM339 (SEQ ID NO:2) and Pichia pastoris NRRL-Y11430 strain genomic DNA as template. The nucleotide sequence of the Pichia pastoris VPS10-1 genomic region, including upstream homologous fragment, promoter, open reading frame (nucleotides 1610-6238), and downstream homologous fragment is provided in FIGS. 13A-13G and SEQ ID NO:14.

The resulting PCR fragment was cloned into pGLY22b using restriction enzymes SacI and PmeI to generate pGLY5191. The downstream 3′ flanking region was amplified with primers MAM340 (SEQ ID NO:3) and MAM341 (SEQ ID NO:4) and Pichia pastoris NRRL-Y11430 strain genomic DNA as template. The resulting fragment was cloned into pGLY5191 using restriction enzymes SalI and SwaI to generate pGLY5192. Both upstream 5′ and downstream 3′ fragments of pGLY5192 were sequenced to verify fidelity.

The plasmid pGLY5194 was constructed to delete the open reading frame of the VPS10-2 gene (see FIG. 1) and create a yeast strain deficient in vacuolar sorting receptor homolog (Vps10-2p) activity. To generate the vps10-2Δ knock-out plasmid pGLY5194, the upstream 5′ flanking region was first amplified using routine PCR conditions with primers MAM439 (SEQ ID NO:5) and MAM343 (SEQ ID NO:6) and Pichia pastoris NRRL-Y11430 strain genomic DNA as template. The nucleotide sequence of the Pichia pastoris VPS10-2 genomic region, including upstream homologous fragment, promoter, open reading frame (nucleotides 830-4509), and downstream homologous fragment is provided in FIGS. 14A-14E and SEQ ID NO:15.

The resulting fragment was cloned into pGLY22b using restriction enzymes SacI and PmeI to generate pGLY5193. The downstream 3′ flanking region was amplified with primers MAM440 (SEQ ID NO:7) and MAM345 (SEQ ID NO:8) and Pichia pastoris NRRL-Y11430 strain genomic DNA as template. The resulting fragment was cloned into pGLY5193 using restriction enzymes SphI and SwaI to generate pGLY5194. Both upstream and downstream fragments of pGLY5194 were sequenced to verify fidelity.

### Example 5

Generation of a Pichia pastoris Strain Expressing GCSF.

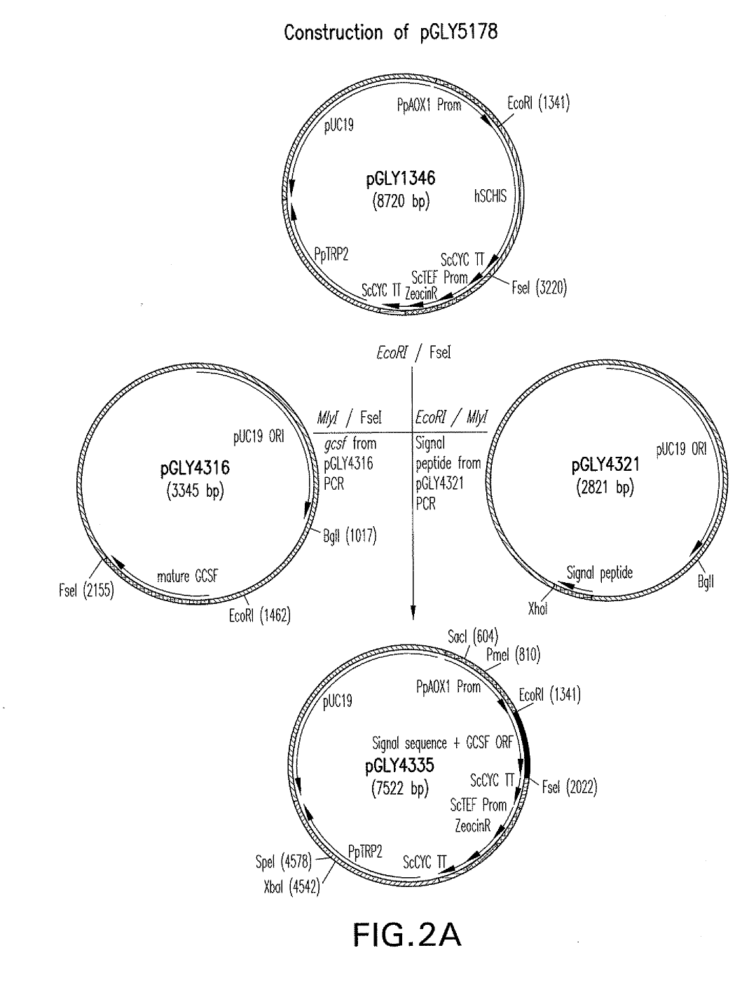

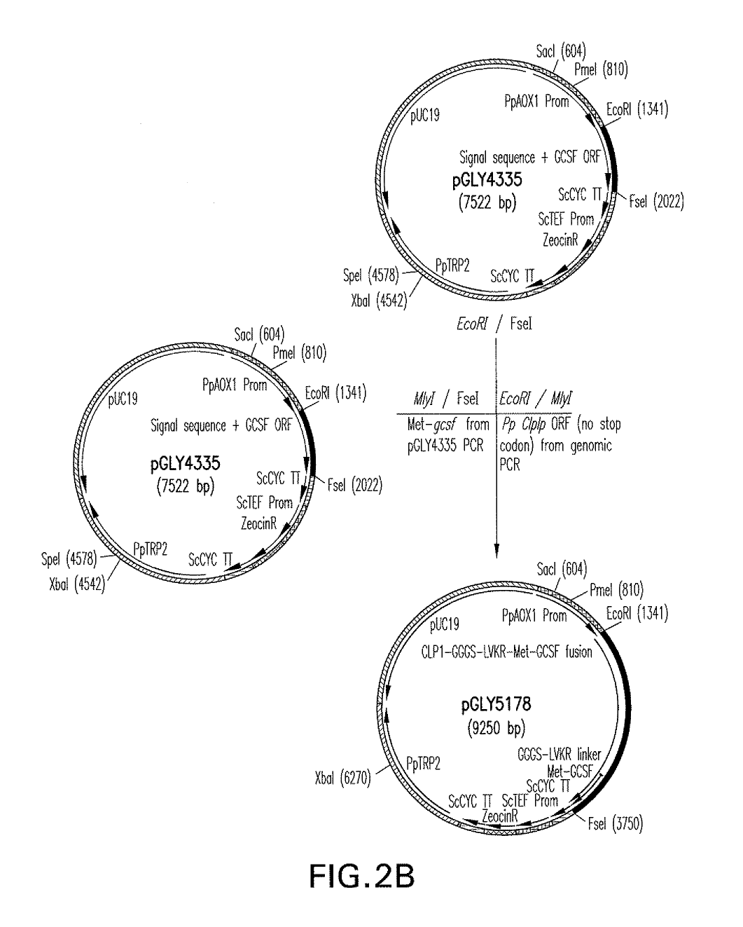

DNA encoding the Homo sapiens granulocyte-cytokine stimulatory factor protein (GCSF, Genbank NP_{—}757373) was synthesized by DNA2.0, Inc. (Menlo Park, Calif.) and inserted into a pUC19 plasmid to make a plasmid designated pGLY4316 (see FIG. 2, SEQ ID NO:16 and SEQ ID NO:168).

A subsequent plasmid was constructed that contained GCSF, amplified using routine PCR conditions from pGLY4316 with primers MAM227 (SEQ ID NO:10) and MAM228 (SEQ ID NO:11). PCR primer MAM27 introduced XhoI and MlyI restriction sites at the 5′ end of the DNA encoding the mature GCSF protein (GCSFp) and an FseI site at the 3′ end of the DNA encoding GCSFp. A DNA fragment encoding a mating factor-IL1β signal peptide (Han et al., Biochem. Biophys. Res. Commun. 18; 337(2):557-62. (2005); Lee et al., Biotechnol Prog. 15(5):884-90 (1999)) that directs the GCSF to the secretory pathway was removed from plasmid pGLY4321 with EcoRI and MlyI digestion. The PCR amplified product was digested with FseI and MlyI and was triple-ligated with the signal peptide encoding fragment into plasmid pGLY1346 digested with EcoRI and FseI to make plasmid pGLY4335 (See FIG. 2) in which the 5′ end of the open reading frame (ORF) encoding the mature GCSF was ligated in frame with the 3′ end of the ORF encoding the signal peptide and which produces a fusion protein in which the N-terminus of the mature GCSF is fused to the C-terminus of the signal peptide.

The GCSF open reading frame was amplified from pGLY4335 by PCR using primers MAM281 (SEQ ID NO:9) and MAM228 (SEQ ID NO:11). The PCR amplified product was digested with the MlyI and FseI restriction enzymes (FIG. 2). Primer MAM281 contains an ATG codon in frame with the GCSF ORF. Thus, the resulting digested amplified PCR product contains an in-frame addition of the ATG translation start codon to the 5′ end of the open reading frame (ORF) encoding the mature GCSF. The resulting fragment contained an in-frame addition of “ATG” nucleotides, which encodes an N-terminal methionine, identical to the Neupogen® (filgrastim, Amgen Inc., Thousand Oaks, Calif.) protein sequence (SEQ ID NO:172).

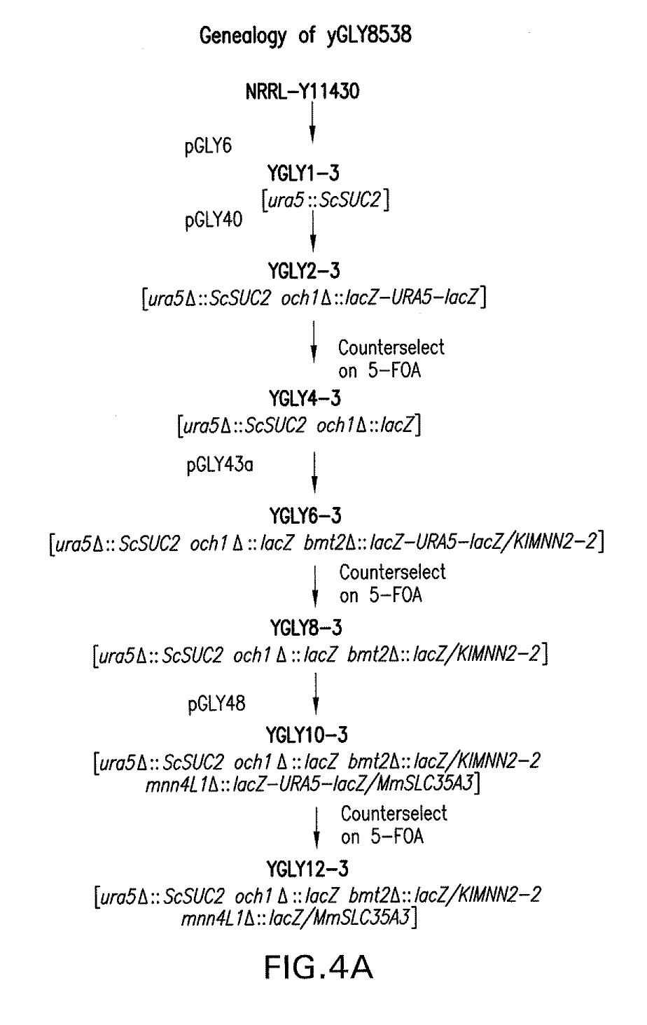

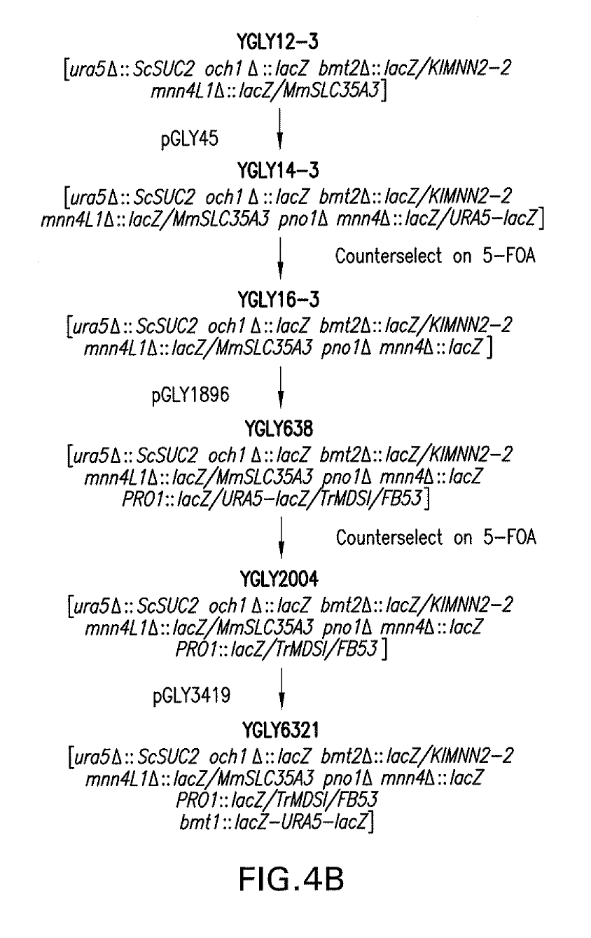

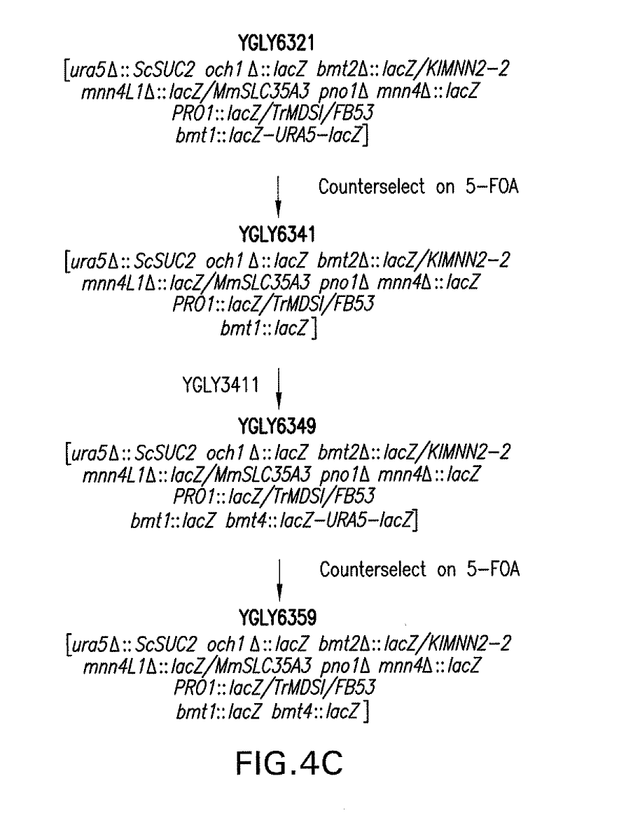

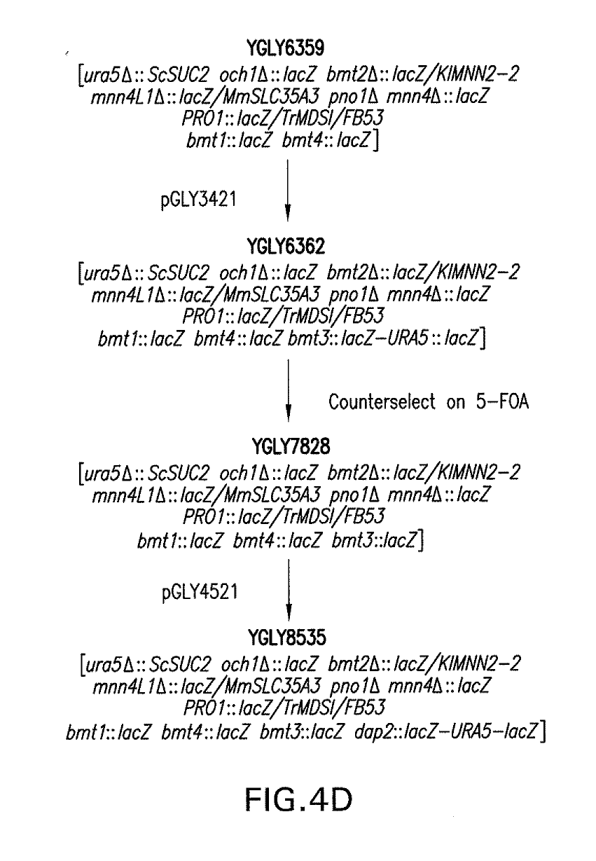

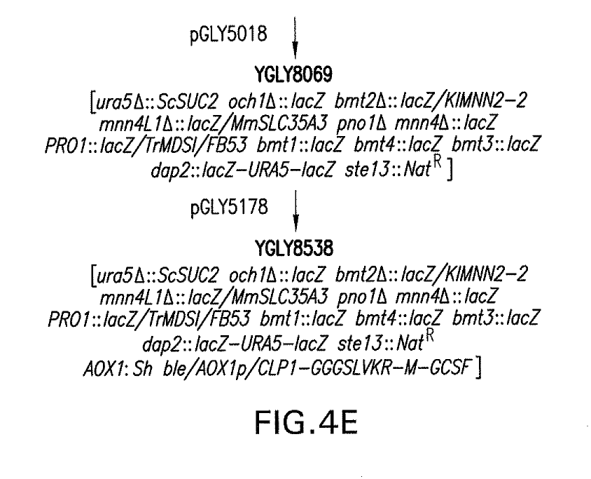

The P. pastoris CLP1 gene (SEQ ID NO:17) was amplified using routine PCR conditions from chromosomal DNA from Pichia pastoris strain NRRL-Y11430 using primers MAM304 (SEQ ID NO:12) and MAM305 (SEQ ID NO:13) and digested with EcoRI and StuI restriction enzymes. A three piece ligation reaction was performed with the EcoRI/StuI digested fragment encoding the P. pastoris CLP1 (PpCLP1), the MlyI/FseI digested fragment encoding the rHuMetGCSF, and plasmid pGLY1346 (digested with EcoRI and FseI) to generate plasmid pGLY5178 as shown in FIG. 2. The insert DNA was sequenced to verify fidelity. Also contained within the pGLY5178 plasmid is the AOX1 (alcohol oxidase) promoter, which drives expression of the complete ORF of the CLP1-GCSF fusion, which includes the complete PpClp1 protein sequence followed by the linker sequence “GGGSLVKR” (SEQ ID NO: 175) and rhMet-GCSF (SEQ ID NOs: 18 and 170). Upon DNA transcription in methanol-containing media, the transcribed mRNA enters the endoplasmic reticulum by the Clp1p signal peptide. The polypeptide is further processed in the Golgi apparatus by the Kex2 protease, which cleaves after the arginine residue in the linker sequence; releasing the two proteins of Clp1 and Met-GCSF to the supernatant fraction (see US 2006/0252069). Protein sequences of processed and secreted Clp1 and Met-GCSF are provided in SEQ ID NO:171 and 172. To express Met-GCSF, plasmid pGLY5178 was linearized with restriction enzyme PmeI and used to transform strain YGLY8069 by roll-in single crossover homologous recombination to generate strain yGLY8538 (see FIG. 4). The strain contains several copies of the expression cassette encoding the rHuMetGCSF integrated into the AOX1 locus. The strain secretes rHuMetGCSF into the medium. The genotype of strain YGLY8538 is ura5Δ::ScSUC2 och1Δ::lacZ bmt2Δ::lacZ/KlMNN2-2 nm n4L IΔ::lacZ/MmSLC35A3 pno1Δ mnn4Δ::lacZ PRO1::lacZ/TrMDSI/FB53 bmt1Δ::lacZ bmt4Δ::lacZ bmt3Δ::lacZ dap2Δ::lacZ-URA5-lacZ ste13Δ::NatR AOX1:Sh ble/AOX1p/CLP1-GGGSLVKR-MetGCSF.

### Example 6

Generation of yGLY8538 Mutant Strains.

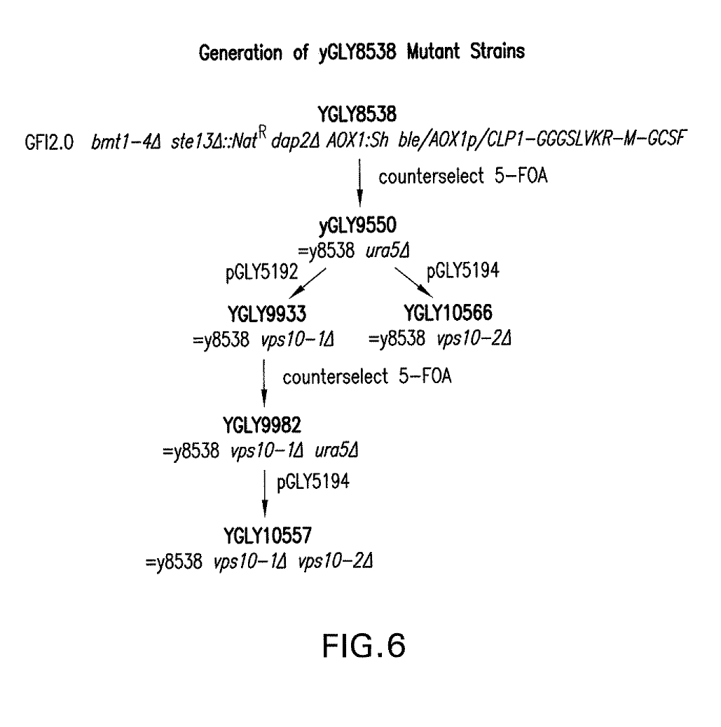

Generation of isogenic mutant yeast strains from yGLY8538 (see FIG. 4) were performed by homologous recombination as described previously (Nett and Gerngross, Yeast 20: 1279-90 (2003)). Parental ura54 strains were transformed with linearized plasmids containing approximately 1000 bp flanking DNA upstream and downstream of the desired open reading frame. Mutant transformants were selected on URA drop-out plates after gaining the lacZ-URA5-lacZ cassette (Nett and Gerngross, supra) and analyzed by PCR to verify the correct genetic profile. The plasmids pGLY5192 (vps10-1Δ) and pGLY5194 (vps10-2Δ) were used for mutagenesis in this study. A flowchart of mutant strain expansion is shown in FIG. 6.

Strains yGLY9933 and yGLY10566 resulted from transformation of yGLY8538 with pGLY5192 (vps10-1Δ) and pGLY 5194 (vps10-2Δ), respectively. In addition, a double knock-out (vps10-1Δ/vps10-2Δ) was constructed by counterselection of yGLY9933 to generate yGLY9982. The plasmid pGLY5194 was electroporated in yGLY9982 to generate the resulting strain yGLY10557 with the vps10-1Δ/vps10-2Δ genotype.

### Example 7

Generation of a Pichia pastoris Strain Expressing TNFRII-Fc.

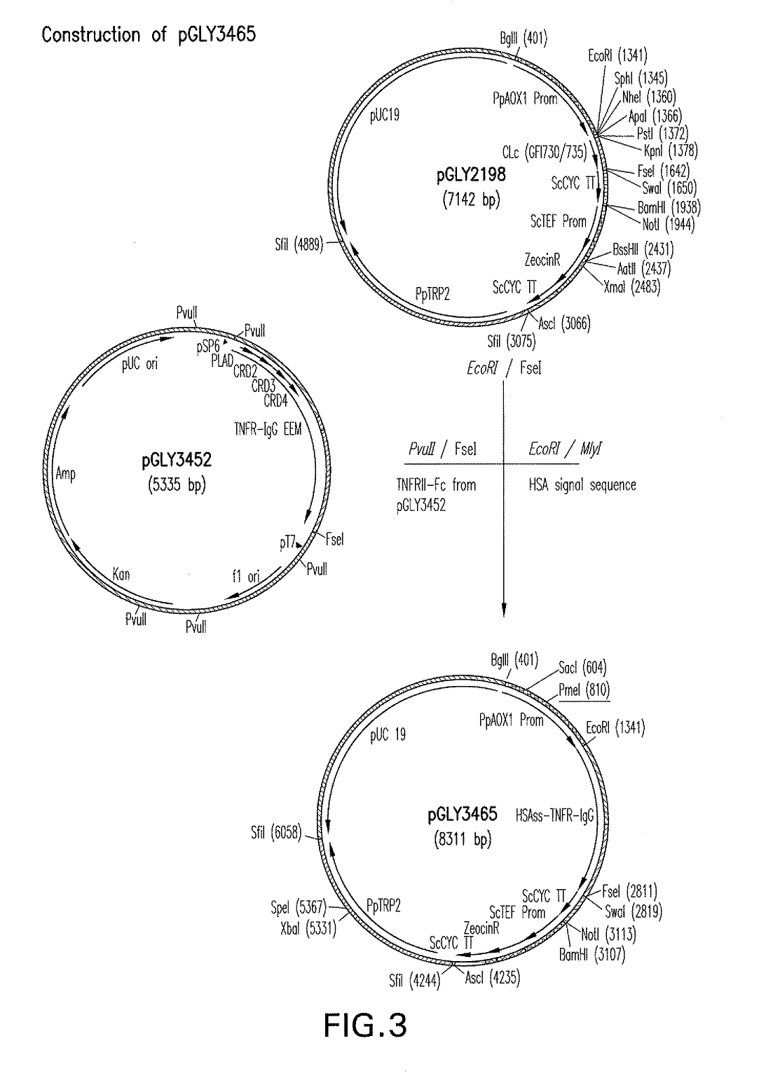

DNA encoding the tumor necrosis factor antagonist TNFRII-Fc (U.S. application Ser. No. 61/256,369) was synthesized by GeneArt AG (Regensburg, Germany,). The full protein was TOPO cloned (Invitrogen) to generate pGLY3452. The TNFRII-Fc open-reading frame was released with PvuII and FseI in order to clone with the USA signal peptide, obtained from synthesized oligonucleotides and digested with EcoRI and MlyI, and plasmid backbone pGLY2198 (EcoRI and FseI). A triple ligation and transformation in E. coli generated expression plasmid pGLY3465 (see FIG. 3). The DNA and protein sequences of TNFRII-Fc are provided in SEQ ID NOs: 19 and 174, respectively.

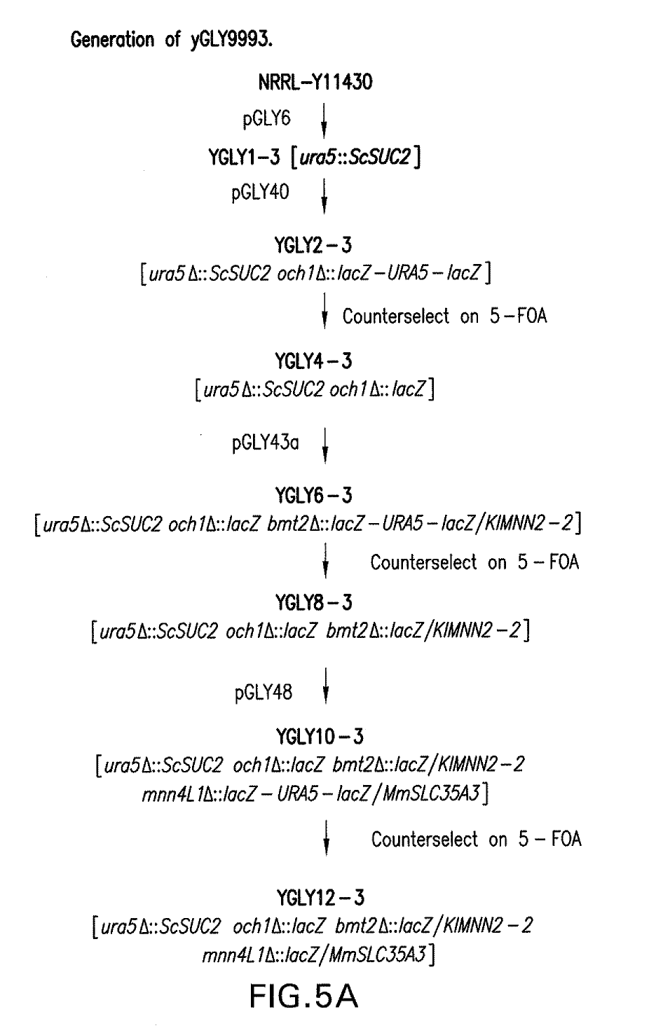

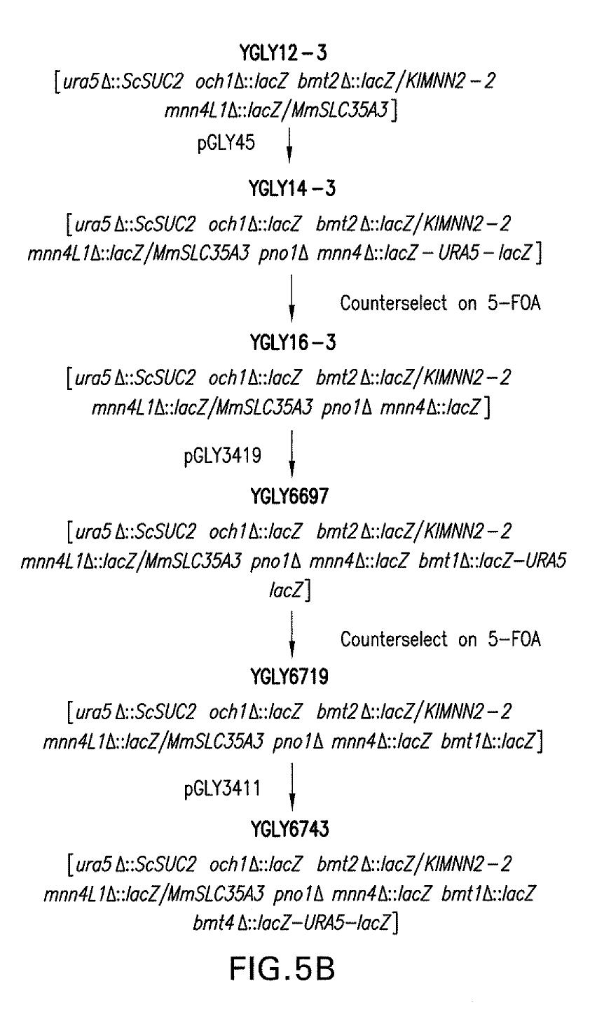

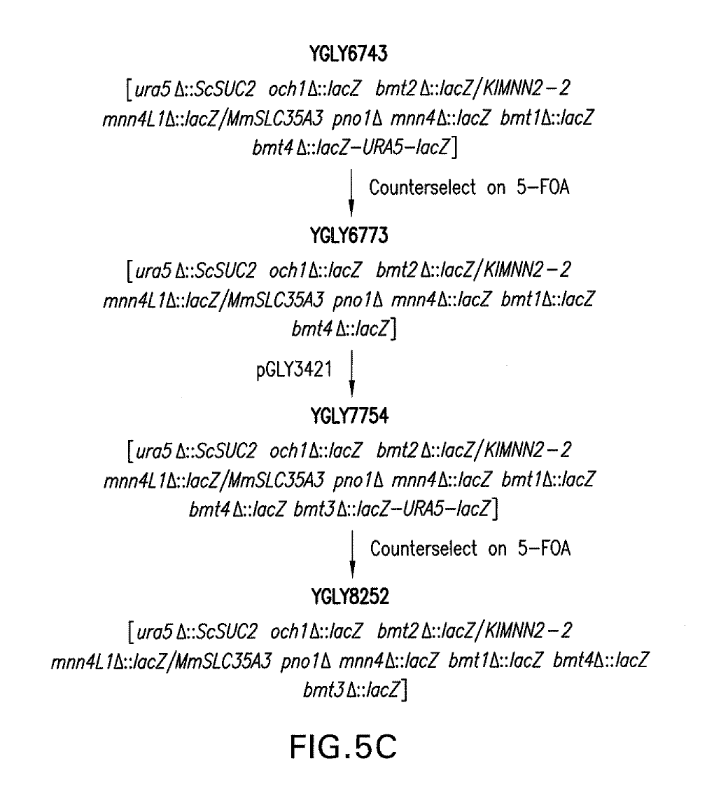

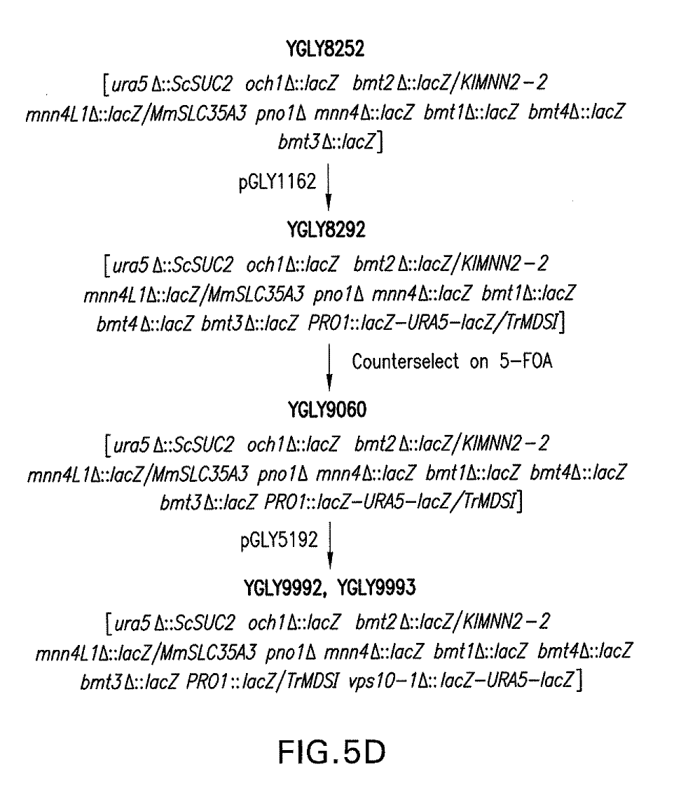

To express TNFRII-Fc, pGLY3456 was linearized with SpeI and electroporated in strains yGLY8292 (VPS10-1), yGLY9992 (vps10-1Δ), and yGLY9993 (vps10-1Δ). The vps10-1d mutant strains, derived from yGLY8292, were generated using plasmid pGLY5192 as shown in FIG. 5.

### Example 8

### Bioreactor Screening and Fermentation Process.

Bioreactor Screenings: Bioreactor Screenings for rhGCSF expression were performed in 0.5 L vessels in a SIXFORS multi-fermentation system (ATR Biotech, Laurel, Md.) under the following conditions: pH at 6.5, 24° C., 0.3 standard liters per minute, and an initial stirrer speed of 550 rpm. The initial working volume was 350 mL, which consisted of 330 mL BMGY medium and 20 mL inoculum. IRIS multi-fermenter software (ATR Biotech, Laurel, Md.) was used to linearly increase the stirrer speed from 550 rpm to 1200 rpm over 10 hours, beginning one hour after inoculation. Seed cultures (200 mL of BMGY in a 1 L baffled flask) were inoculated directly from agar plates. The seed flasks were incubated for 72 hours at 24° C. to reach optical densities (0D₆₀₀) between 95 and 100. The fermenters were inoculated with 200 mL stationary phase flask cultures that were concentrated to 20 mL by centrifugation. The batch phase ended on completion of the initial charge glycerol (18-24 h) fermentation and was followed by a second batch phase that was initiated by the addition of 17 mL of glycerol feed solution (50% [w/w] glycerol, 5 mg/L Biotin, 12.5 mL/L PTM1 salts (65 g/L FeSO₄.7H₂O, 20 g/L ZnCl₂, 9 g/L H₂SO₄, 6 g/L CuSO₄.5H₂O, 5 g/L H₂SO₄, 3 g/L MnSO₄.7H₂O, 500 mg/L CoCl₂.6H₂O, 200 mg/L NaMoO₄.2H₂O, 200 mg/L biotin, 80 mg/L NaI, 20 mg/L H₃BO₄)). Upon completion of the second batch phase, as signaled by a spike in dissolved oxygen, the induction phase was initiated by feeding a methanol feed solution (100% MeOH 5 mg/L biotin, 12.5 mL/L PTM1) at 0.6 g/h for 32-40 hours. The cultivation was harvested by centrifugation.

Platform Fermentation Process:

Bioreactor cultivations were done in 3 L and 15 L glass bioreactors (Applikon, Foster City, Calif.) and a 40L stainless steel, steam in place bioreactor (Applikon, Foster City, Calif.). Seed cultures were prepared by inoculating BMGY media directly with frozen stock vials at a 1% volumetric ratio. Seed flasks were incubated at 24° C. for 48 hours to obtain an optical density (0D₆₀₀) of 20±5 to ensure that cells are growing exponentially upon transfer. The cultivation medium contained 40 g glycerol, 18.2 g sorbitol, 2.3 g K₂HPO₄, 11.9 g KH₂PO₄, 10 g yeast extract (BD, Franklin Lakes, N.J.), 20 g peptone (BD, Franklin Lakes, N.J.), 4×10⁻³ g biotin and 13.4 g Yeast Nitrogen Base (BD, Franklin Lakes, N.J.) per liter. The bioreactor was inoculated with a 10% volumetric ratio of seed to initial media. Cultivations were done in fed-batch mode under the following conditions: temperature set at 24±0.5° C., pH controlled to 6.5±0.1 with NH₄OH, dissolved oxygen was maintained at 1.7±0.1 mg/L by cascading agitation rate on the addition of O₂. The airflow rate was maintained at 0.7 vvm. After depletion of the initial charge glycerol (40 g/L), a 50% (w/w) glycerol solution (containing 12.5 ml/L of PTM2 salts and 12.5 ml/L of 25XBiotin) was fed exponentially at a rate of 0.08 h⁻¹ starting at 5.33 g/L/hr (50% of the maximum growth rate) for eight hours. Induction was initiated after a 30 minute starvation phase when methanol (containing 12.5 ml/L of PTM2 salts and 12.5 ml/L of 25XBiotin) was fed exponentially to maintain a specific growth rate of 0.01 h⁻¹ starting at 2 g/L/hr.

For YGLY8538, rHuMetGCSF was generated using high methanol feed rate (ramped the methanol feed rate from 2.33 g/L/hr to 6.33 g/L/hr in a 6 hr period and maintained at 6.33 g/L/hr for the entire course of induction) and by adding 0.68 g/L of Tween 80 into the methanol. Fermentation pH was reduced to 5.0 as a process improvement for this and the following strains.

For YGLY9933, the high methanol feed rate, 0.68 g/L Tween 80, and fermentation pH 5.0 was utilized.

### Example 9

### Deep-Well Induction Plates.

Titer improvement of TNFRII-Fc was determined using deep-well plate screening. Transformants were inoculated to 600 μL BMGY and grown at 24° C. for two days in a micro-plate shaker at 840 rpm. The resulting 50 μL seed culture was transferred to two 96-well plates containing 600 μL fresh BMGY per well and incubated for two days at the same culture conditions as above. The two expansion plates were combined to one plate, and then centrifuged for 5 minutes at 1000 rpm. The cell pellets were induced in 600 μL BMMY per well for two days and then the centrifuged 400 μL clear supernatant was analyzed by ELISA.

### Example 10

### GCSF Titer Determination.

Cleared supernatant fractions were assayed for GCSF titer with a standard ELISA protocol. Briefly, polyclonal anti-GSCF (R&D Systems®, Minneapolis, Minn., Cat#MAB214) was coated onto a 96 well high binding plate (Corning®, Corning, N.Y., Cat#3922), blocked, and washed. An rhGCSF protein standard (R&D Systems®, Cat. #214-CS) and serial dilutions of cell-free supernatant fluid were applied to the above plate and incubated for 1 hour. Following a washing step, monoclonal anti-GCSF (R&D Systems®, Cat#AB-214-NA) was added to the plate and incubated for 1 hour. After washing, an alkaline phosphatase-conjugated goat anti-mouse IgG Fc (Thermo Fisher Scientific®, Waltham, Mass., Cat#31325) was added and incubated for 1 hour. The plate was washed and the fluorescent detection reagent 4-MUPS was added and incubated in the absence of light. Fluorescent intensities were measured on a TECAN fluorimeter (Tecan Group, Ltd., Männedorf, Switzerland) with 340 nm excitation and 465 nm emission properties.

### Example 11

### TNFRII-Fc Titer Determination.

Cleared supernatant fractions were assayed for TNFRII-Fc titer with a standard ELISA protocol. Briefly, monoclonal anti-human sTNFRII/TNFRSF1B (R&D Systems®, Cat#MAB726) was coated onto a 96 well high binding plate (Corning®, Cat#3922), blocked, and washed. A TNFRII-Fc protein standard (commercial ENBREL®, Amgen, Thousand Oaks, Calif.) and serial dilutions of cell-free supernatant fluid were applied to the above plate and incubated for 1 hour. Following a washing step, polyclonal anti-human sTNFRII/TNFRSF1B (R&D Systems®, Cat#AB-26-PB) was added to the plate and incubated for 1 hour. After washing, an alkaline phosphatase-conjugated donkey anti-goat IgG (Santa Cruz®, Cat#SC-2022) was added and incubated for 1 hour. The plate was washed and the fluorescent detection reagent 4-MUPS was added and incubated in the absence of light. Fluorescent intensities were measured on a TECAN fluorimeter with 340 nm excitation and 465 nm emission properties.

### Example 12

### Cell Lysis Determination.

Cell lysis was measured by assaying the amount of double-stranded DNA in the fermentation supernatant. The Quant-iT™ PicoGreen® assay kit (Invitrogen Corp., Carlsbad, Calif.) was used to assay for dsDNA according to the manufacturer's suggestions.

### Results:

### Example 13

Human GCSF and TNFRII-Fc Contain a Canonical Vps10 Binding Sequence.

In Saccharomyces cerevisiae, the Vps 10 (also known as Pep1 or Vpt1) receptor is responsible for binding pro-carboxypeptidase y (pro-Cpy, also known as Pre1) via a “QRPL-like” sorting signal (Gln²⁴-Arg-Pro-Leu²⁷, SEQ ID NO:176) and transporting pro-Cpy to the vacuole (Marcusson et al. Cell 77: 579-86 (1994); Valls et al. Cell 48: 887-97 (1987)). Previous studies have focused on the sorting of Cpy in S. cerevisiae to examine binding interactions. These studies identified two regions of the Vps10 luminal receptor domain, each with distinct ligand binding affinities (Jorgensen et al. Eur J Biochem 260: 461-9 (1999); Cereghino et al. Mol Biol Cell 6: 1089-102 (1995); and Cooper & Stevens J Cell Biol 133: 529-41 (1996)). Additionally, van voorst and colleagues (J Biol Chem 271: 841-6 (1996)) performed mutagenesis of the Cpy “QRPL” peptide near the amino terminus to determine the requirement for sequence conservation to the efficiency of vacuolar sorting. Their analysis revealed that, other than at position Gln²⁴, multiple substitutions could be made without affecting the interaction with Vps 10 or leading to missorting. The S. cerevisiae Vps10 receptor was also shown to interact with recombinant proteins, such as E. coli β-lactamase, in an unknown mechanism not involving a “QRPL-like” sorting domain (Holkeri and Makarow, FEBS Lett 429: 162-6 (1998)). In S. cerevisiae, previous research identified three additional homologs of Vps10 (Vth1, Vth2, YNR065c, see FIG. 12) with potential sorting activity (Cooper & Stevens J Cell Biol 133: 529-41 (1996); Westphal et al. J Biol Chem 271(20):11865-70 (1996); Tarassov K, et al. Science 320(5882):1465-70 (2008)).

We identified sequences near the amino termini of recombinant human granulocyte-colony stimulating factor (rhGCSF) and TNFRII-Fc with characteristics of a Vps10 sorting sequence (van Voorst et al (1996), supra). These sequences are “QSFL” (SEQ ID NO:177) for GCSF (see Genbank NP_{—}757373 or SEQ ID NO:168) and “QVAF” (SEQ ID NO:178) for TNFRII-Fc (see SEQ ID NO:174). As shown in Table 1, below, each of the four amino acid positions in the putative Vps10 binding domain of rhGCSF and TNFRII-Fc were compared to previous mutagenesis results for Cpy vacuolar targeting (Tamada et al. Proc Natl Acad Sci USA 103: 3135-40, 11 (2006); van Voorst et al. (1996), supra). When the amino acids of the sorting peptide in rhGCSF and TNFRII-Fc were compared to the respective mutated pro-Cpy protein, all mutations were reported to reveal no less than 85% activity (see FIG. 3 of van Voorst et al. (1996), supra). These data indicate the sorting peptides in rhGCSF and TNFRII-Fc would likely bind to the Vps10 receptor if surface exposed and direct the recombinant protein to the yeast vacuole.

TABLE 1
Possible Vps 10p-binding Motifs

| | | % Relative |
|---|---|---|
| | N-terminal | Efficiency to |
| Protein | Sequence | S.c. Cpy “QRPL” |
| hGCSF | ^{↓}TPLGPASSLPQSFLLK | 100-85-90-100 |
| | (SEQ ID NO: 179) | |
| TNFRII-Fc | ^{↓}LPAQVAFTP | 100-100-90-100 |
| | (SEQ ID NO: 180) | |

Furthermore, both peptides map to a surfaced-exposed region of the respective protein capable of interacting with Vps10 (Hill et al. Proc Natl Acad Sci USA 90: 5167-71 (1993), Tamada et al. (2006), supra). Based on the likelihood of GCSF and TNFRII-FC binding to the Vps 10 receptor via N-terminal sorting sequences and their surface exposure, we hypothesized that mutations in the P.p. VPS10 homologs would improve secretory yields of rhGCSF and TNFRII-Fc by eliminating vacuolar sorting.

### Example 14

Homologs of Vps10 in P. pastoris.

A TBlastN search of the genomic DNA sequence of Pichia pastoris revealed two gene homologs of VPS10 in Pichia pastoris, denoted VPS10-1 and VPS10-2 (see Example 3). A comparison of S. cerevisiae and P. pastoris Vps 10 protein homologs is shown in FIG. 12. Whereas S.c. Vps10 is 1579aa, P.p. Vps10-1 is 29.99% identical (1542aa) and P.p. Vps10-2 is 25.4% identical (1502aa). Alignment between P.p Vps10-1 and Vps10-2 proteins revealed 41.0% similarity and 26.8% identity. Similar to S.c. Vps10, both P. pastoris proteins have a predicted N-terminal signal peptide for entry into the endoplasmic reticulum, two C-terminal rich regions, and a single predicted transmembrane domain near the C-terminus (Horazdovsky et al. Curr Opin Cell Biol 7: 544-51 (1995)) (data not shown).

As discussed above, alignments of the P. pastoris Vps10 proteins (Vps10-1 and Vps10-p) to the S. cerevisiae Vps10 demonstrated a relatively low 37-43 percent identity; whereas alignments of the other S. cerevisiae Vps10 homologs (Vth1p, Vth2p, YNR065C) to S. cerevisiae Vps10 demonstrated a 58-75 percent identity (FIG. 12). Therefore, based on sequence analysis alone, it could not be determined whether the two P. pastoris Vps10 homologs will function similarly as the S. cerevisiae Vps10.

Additional fungal Vps10 homologs were identified from GenBank® (National Center for Biotechnology Information (NCBI), Bethesda, Md.) and aligned with S. cerevisiae Vps10 (FIG. 12). The following GenBank® accessions were designated Vps10 homologs: Aspergillus niger (CAK38444, SEQ ID NO:26, FIG. 18), Schizosaccharomyces pombe (CAA16914.1, SEQ ID NO:27, FIG. 19), Candida albicans (EAK91536, SEQ ID NO:28, FIG. 20), Candida glabrata (CAG60842.1, SEQ ID NO:29, FIG. 21), Pichia stipitis (NC_{—}009068.1, SEQ ID NO:30, FIG. 22), Debaryomyces hansenii (XP_{—}002770499., SEQ ID NO:181, FIG. 23), and Kluyveromyces lactis (XP_{—}454425, SEQ ID NO:182, FIG. 24). Data from S. pombe indicates that while the Vps10 receptor has only 23.6 percent identity to S. cerevisiae Vps10, it exhibits similar functions (Iwaki et al. Microbiology 152: 1523-32 (2006); Takegawa et al. Cell Struct Funct 28: 399-417 (2003); Takegawa et al. Curr Genet. 42: 252-9 (2003)). In all, the bioinformatic data suggests the two P. pastoris Vps10 homologs may have a function that is similar to the S. cerevisiae Vps 10 receptor.

### Example 15

Vps10-1 Activity Reduces rhGCSF Titer.

The parental rhGCSF expression strain, yGLY8538, utilizes the AOX1 promoter to transcribe GCSF. The parental strain was counterselected using 5-fluoroorotic acid (5-FOA) to generate mutant strains (see FIGS. 6 and 11B). Isogenic mutants (URA5+) of P.p. vps10-1Δ (yGLY9933) and vps10-2Δ (yGLY10566) were generated by electroporation of plasmids pGLY5192 and pGLY5194, respectively (see Examples 1-11, FIG. 1). The effects of vps10-1Δ and vps10-2Δ mutations on rhGCSF secretion were determined using Sixfors fermentors (ATR Biotech, Laurel, Md.) and a GCSF ELISA assay (see Example 10).

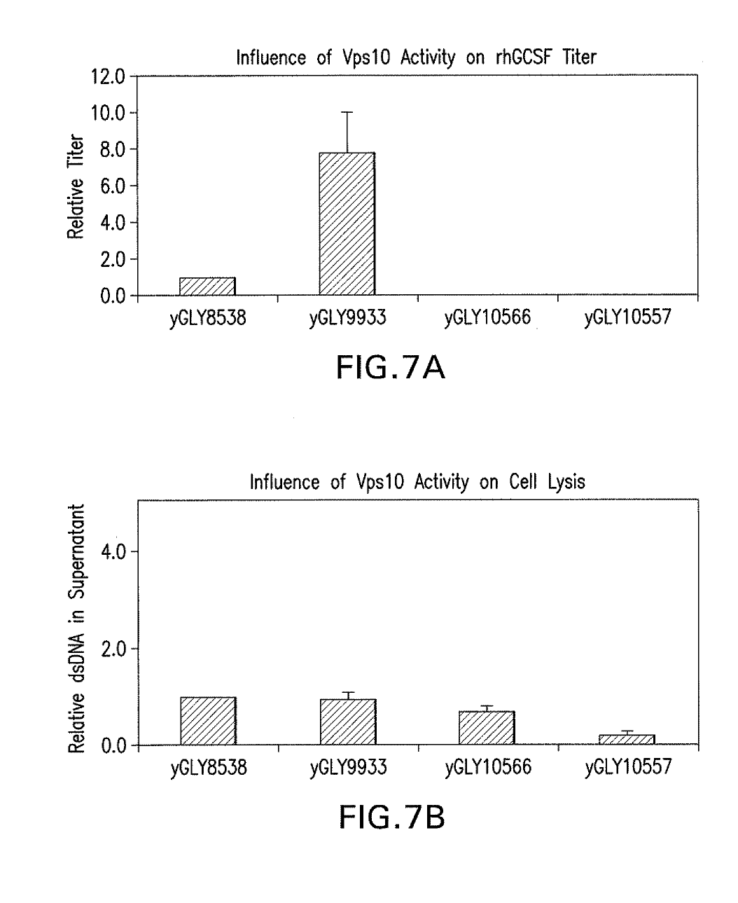

Results revealed that the vps10-1Δ mutant yGLY9933 secreted over seven times as much rhGCSF relative to yGLY8538 (FIG. 7A). Surprisingly, the vps10-2Δ mutant yGLY10566 did not secrete any detectable rhGSCF. Fermentation supernatant from yGLY10566 was subjected to SDS-PAGE analysis to reveal a dramatic ablation of total secreted protein (data not shown). These results indicate that the functions of Vps10-1 and Vps10-2 are not redundant in their interactions with rhGCSF. Titer results from the vps10-1Δ vps10-2Δ double mutant (yGLY10557) demonstrated the vps10-2Δ mutation was dominant over vps10-1Δ mutation, whereby rhGCSF (FIG. 7A) and the majority of all secreted proteins were drastically reduced (not shown). These fermentation samples were also assayed for cell lysis as measured by double-stranded DNA released from cells into the supernatant fraction. Because we have not seen any disclosures of yeast vps10 mutants in fermentation conditions, it was possible that during high biomass fermentation conditions, cell fitness could become compromised if normal vacuolar function was altered. If this were to occur, cells may lyse and release double-stranded DNA into the supernatant fraction. However, data shown in FIG. 7B indicate mutations in vps10-1Δ and/or vps10-2Δ do not induce cell lysis.

### Example 16

### Vps10-1 Activity Reduces TNFRII-Fc Titer.

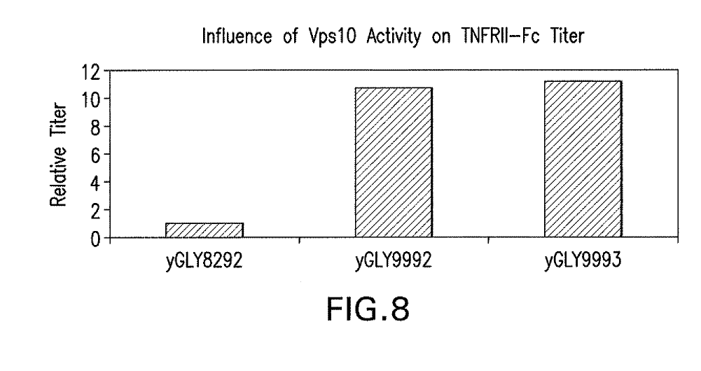

Since TNFRII-Fc also contains a putative Vps10 binding motif in the N-terminus, we transformed the expression vector pGLY3465 in cell lineages with and without functional Vps10-1. At least eleven independent transformants were induced for protein expression. ELISA titers were individually calculated, then averaged for each host strain. The relative ELISA titer was determined from average ELISA titers of each host strain divided by the average ELISA titers of the wild-type parental strain yGLY8292. (FIG. 8) This data clearly shows that the vps10-1Δ mutant strains (yGLY9992 and yGLY9993) exhibit approximately ten-fold higher TNFRII-Fc secretion levels than the parental wild-type strain yGLY8292.

### Example 17

Model of Pichia pastoris Vps10-1 function.

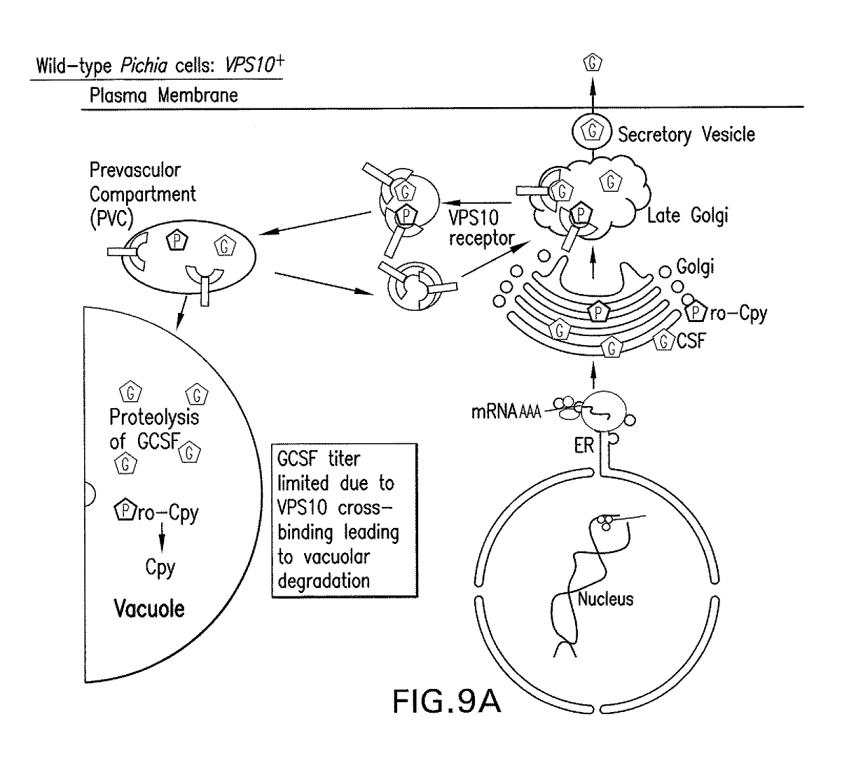

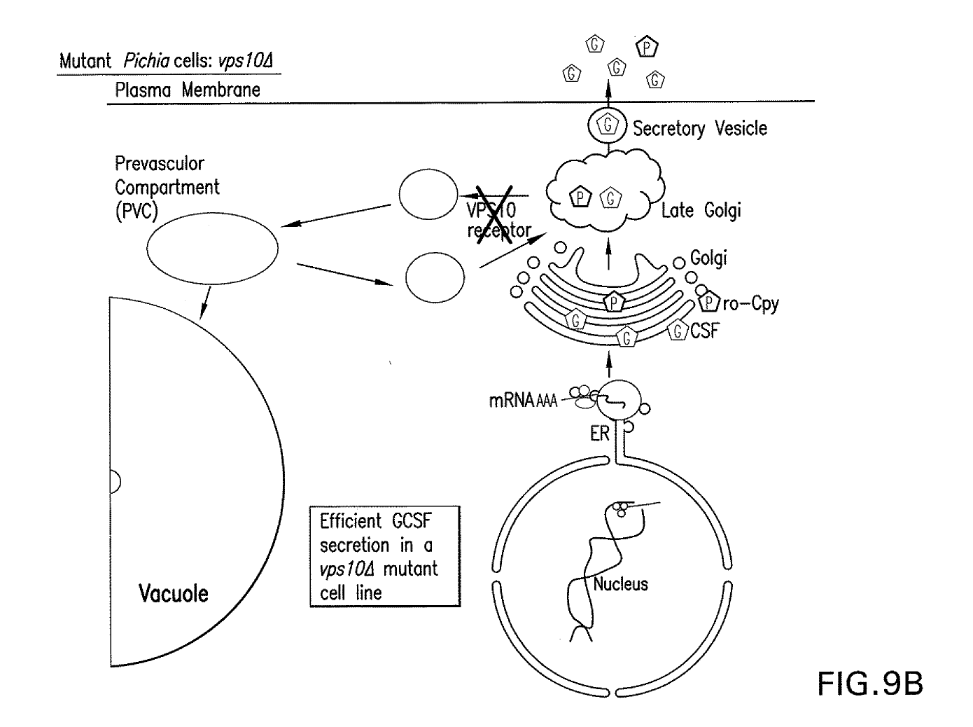

The data indicates Vps 10-1 is capable of interacting with recombinant proteins transiting the secretory pathway in Pichia pastoris. FIG. 9A illustrates the altered delivery of a recombinant protein to the vacuole with normal function of Vps10-1, using rhGCSF as a model protein. In contrast, FIG. 9B illustrates the efficient secretion of rhGCSF into the supernatant fraction when activity of Vps10-1 is eliminated or reduced. The reduction of Vps10-1 activity thereby renders cells more productive at recombinant protein secretion.

1. A Pichia pastoris cell lacking vacuolar sorting activity or having reduced vacuolar sorting activity relative to a wild-type Pichia pastoris cell, wherein the host cell comprises a functional deletion of a vacuolar protein sorting receptor 10-1 (VPS10-1).

2. The Pichia pastoris cell of claim 1; wherein the cell comprises an expression vector which comprises a sequence of nucleotides that encodes a heterologous protein.

3. The Pichia pastoris cell of claim 2, wherein the heterologous protein is a glycoprotein.

4. The Pichia pastoris cell of claim 3, wherein the cell is modified to express a glycoprotein in which the glycosylation pattern is human-like.

5. The Pichia pastoris cell of claim 1, wherein a gene encoding VPS10-1 is deleted and a gene encoding VPS10-2 is not deleted.

6. The Pichia pastoris cell of claim 1, wherein a gene encoding VPS10-1 comprises a mutation that renders the encoded Vps10-1 protein nonfunctional or incapable of vacuolar sorting activity.

7. The Pichia pastoris cell of claim 1, wherein the functional deletion of Vps10-1 activity comprises an alteration selected from the group consisting of: deletion or disruption of upstream or downstream regulatory sequences of the VPS10-1 gene, abrogation of vacuolar sorting activity by means of a chemical, peptide or protein inhibitor of Vps10-1 protein, abrogation of vacuolar sorting activity by means of a nucleic acid-based expression inhibitor and abrogation of vacuolar sorting activity by means of a transcription inhibitor.

8. A method for producing a recombinant protein in a yeast or fungal host cell comprising:
a. transforming a genetically modified yeast or fungal cell with an expression vector encoding the protein to produce a host cell, wherein the genetically modified yeast or fungal cell lacks vacuolar sorting activity or has decreased vacuolar sorting activity relative to an unmodified yeast or fungal cell of the same species;
b. culturing the transformed yeast or fungal host cell in a medium under conditions which induce expression of the protein in fermentation conditions; and
c. isolating the protein from the transformed host cell or culture medium.

9. The method of claim 8, wherein the yeast or fungal host cell is selected from the group consisting of: Pichia pastoris, Saccharomyces cerevisiae, Aspergillus niger, Schizosaccharomyces pombe, Candida albicans, Candida glabrata, Pichia stipitis, Debaryomyces hansenii, Kluyveromyces lactis, and Hansenula polymolpha.

10. The method of claim 8 or 9, wherein vacuolar sorting activity has been eliminated or reduced by deletion or disruption of a gene encoding VPS10 or a VPS10 homolog from the yeast or fungal cell genome.

11. The method of claim 10, wherein the yeast or fungal host cell is Pichia pastoris.

12. The method of claim 11, wherein the VPS10 homolog VPS10-1 is deleted.

13. A method for producing a recombinant protein in a Pichia host cell comprising:
a. transforming a genetically modified Pichia cell with an expression vector encoding the protein to produce a host cell, wherein the genetically modified Pichia cell lacks vacuolar sorting activity or has decreased vacuolar sorting activity relative to an unmodified Pichia cell of the same species;
b. culturing the transformed Pichia host cell in a medium under conditions which induce expression of the protein; and
c. isolating the protein from the transformed host cell or culture medium.

14. The method of claim 13, wherein the host cell is a Pichia pastoris host cell.

15. The method of claim 14, wherein the genetically modified Pichia pastoris cell comprises a deletion of VPS10-1.

16. The method of claim 8, wherein the genetically modified host cell comprises an alteration of the cytoplasmic domain of Vps10 or the Vps10 homolog that alters its normal trafficking pattern.

17. The method of claim 8, wherein vacuolar sorting activity is reduced or eliminated by deletion or disruption of one or more genes that are associated with the CPY vacuolar sorting pathway, wherein the one or more genes encode a protein selected from the group consisting of: Gga1, Gga2, Mvp1, Pep12, Vps1, Vps8, Vps9, Vps15, Vps21, Vps19, Vps34, Vps38, Vps45, and Vti1.

18. The method of claim 8, wherein vacuolar sorting activity is reduced or eliminated by deletion or disruption of one or more genes that encode a protein associated with recycling of Vps10 to the late Golgi, wherein the one or more genes encode a protein selected from the group consisting of: Grd19, Rgp1, Ric1, Vps5, Vps17, Vps26, Vps29, Vps30, Vps35, Vps51, Vps52, Vps53, and Vps54.

19. The method of claim 8, wherein vacuolar sorting activity is reduced or eliminated by deletion or disruption of one or more genes that encode a protein associated with MVB function, wherein the one or more genes encode a protein selected from the group consisting of: Ccz1, Fab1, Hse1, Mrl1, Vam3, Vps2, Vps3, Vps4, Vps11, Vps13, Vps16, Vps18, Vps20, Vps22, Vps23, Vps24, Vps25, Vps27, Vps28, Vps31, Vps32, Vps33, Vps36, Vps37, Vps39, Vps41, Vps43, Vps44, Vps46, Vta1, and Ypt7.

20. The method of claim 8, wherein the expression vector encodes a glycoprotein and wherein the modified host cell has been further modified to express a glycoprotein in which the glycosylation pattern is human-like.
